(19) United States
(12) Patent Application Publication
(10) Pub. No.: US 2026/0219705 A1
(43) Pub. Date: Jul. 30, 2026

(54) SMART RINGS HAVING COMPACT FORM FACTORS

(71) Applicant: Oura Health Oy, Oulu (FI)

(72) Inventors: Jukka-Tapani Mäkinen, Oulu (FI); Miika Kanste, Oulu (FI)

(73) Assignee: Oura Health Oy, Oulu (FI)

(21) Appl. No.: 19/038,863

(22) Filed: Jan. 28, 2025

(51) Int. Cl. G06F 1/16; H05K 1/181; H05K 1/189; G06F 3/01

(52) U.S. Cl. CPC G06F 1/163; G06F 1/1635; G06F 1/1698; H05K 1/181; H05K 1/189; G06F 3/015; H05K 2201/10106

(57) ABSTRACT

Systems and methods are disclosed for smart rings having compact form factors. In one embodiment, a wearable device can include a printed circuit board having a first portion and a second portion, a first light emitting diode (LED) disposed on the first portion of the printed circuit board, the first LED configured to output green light, a second LED disposed on the first portion of the printed circuit board, a third LED disposed on the second portion of the printed circuit board, the third LED configured to output red light, and a photodiode disposed on the first portion of the printed circuit board, the photodiode configured to detect light output from at least one of the first LED, the second LED, or the third LED. The first LED, the second LED, and the third LED are directly attached to the printed circuit board.

### BACKGROUND

Users of wearable devices may utilize such devices for various purposes, such as tracking movement, biometrics, activity levels, and so forth. Wearable devices in different form factors, such as rings, watches, and the like may be used. Different form factors have different technical challenges related to device performance. For example, wearable devices in a watch form factor may not consistently be in contact with a user's skin, whereas wearable devices in a ring form factor may be uncomfortable if too bulky. Moreover, for wearable devices in a ring form factor, the size, shape, weight, and other physical characteristics of the wearable device may impact a comfort level for the user wearing the ring device. For example, users may not prefer bulky and/or heavy wearable ring devices. Accordingly, wearable devices, such as wearable ring devices, having compact form factors may be desired.

### BRIEF DESCRIPTION OF THE DRAWINGS

FIG. 1 is a hybrid schematic illustration of an example use case for smart rings having compact form factors in accordance with one or more embodiments of the disclosure.

FIG. 2 is a schematic illustration of an example difference in form factor for a wearable ring device having a compact form factor in accordance with one or more embodiments of the disclosure.

FIG. 3 is a schematic illustration of an example printed circuit board with chip-level optoelectronic components and reflective structures for use with a smart ring having a compact form factor in accordance with one or more embodiments of the disclosure.

FIG. 4 depicts an example improved sensor function for smart rings having compact form factors relative to a typical wearable device in accordance with one or more embodiments of the disclosure.

FIG. 5 is a schematic illustration of an additional example improved sensor function for smart rings having compact form factors in accordance with one or more example embodiments of the disclosure.

FIG. 6 is a schematic illustration of an additional example improved sensor function for smart rings having compact form factors in accordance with one or more example embodiments of the disclosure.

FIGS. 7A-7B schematically illustrate example computer system architecture in accordance with one or more embodiments of the disclosure.

The detailed description is set forth with reference to the accompanying drawings. The drawings are provided for purposes of illustration only and merely depict example embodiments of the disclosure. The drawings are provided to facilitate understanding of the disclosure and shall not be deemed to limit the breadth, scope, or applicability of the disclosure. The use of the same reference numerals indicates similar, but not necessarily the same or identical components. Different reference numerals may be used to identify similar components. Various embodiments may utilize elements or components other than those illustrated in the drawings, and some elements and/or components may not be present in various embodiments. The use of singular terminology to describe a component or element may, depending on the context, encompass a plural number of such components or elements and vice versa.

### DETAILED DESCRIPTION

### OVERVIEW

Users may utilize smart or active wearable devices in a ring form factor to track various user data, such as the user's activity level, sleep metrics, stress levels, physiological metrics, and/or other data. Such ring devices may be worn on a user's finger. A ring form factor may be less intrusive or bothersome to a user relative to larger form factors, such as watches, bands, etc. Moreover, wearable devices in a ring form factor may be aesthetically pleasing to users.

Users may prefer wearable ring devices that have compact form factors, so as to increase comfort and improve aesthetic appearance. However, reducing the form factor of a wearable ring device may impact certain functionality of the wearable device, and may be limited by the size of certain electronic components included in the wearable ring device. For example, heart rate measurements, or photoplethysmography (PPG) measurements, may be determined using one or more PPG sensors that rely on light-based measurements. As form factors of wearable ring devices become compact, technical challenges and complications can result.

Embodiments of the disclosure include wearable devices, such as finger-worn wearable ring devices, having compact form factors, while providing various functionality desired by users. In some embodiments, sensor performance and/or wearable ring device functionality itself may be improved. For example, some embodiments have compact form factors and also provide the ability to determine blood glucose levels and/or other measurements not determined by typical wearable devices. Embodiments may therefore provide improved functionality, maintained and/or improved sensor performance, and aesthetic design optionality in a compact form factor relative to typical wearable devices.

Some embodiments include chip-level optoelectronic components, which reduce an internal footprint of such components, thereby allowing for reducing external form factor. Chip- level optoelectronic components may be electronic components that are directly attached to a printed circuit board or other substrate. In contrast, typical optoelectronic components may be formed in packages with support structures (e.g., a housing disposed about the optoelectronic component that supports the component, etc.), where the package itself is mounted to a printed circuit board or other substrate. Such packages consume valuable space inside a wearable device. Chip-level optoelectronic components may be devoid of support structures and/or packages, in that the components themselves are not coupled directly to a package or support structure. Instead, the chip-level component may be packageless and attached or mounted directly to the printed circuit board or other substrate. Examples of chip-level optoelectronic components include, but are not limited to, light emitting diodes, laser diodes, photodetectors or photodiodes, and the like. Embodiments may include one or more chip-level components, including chip-level light emitting assemblies with more than one light emitting chip, so as to be configured to output lights of different colors using a single assembly.

Chip-level optoelectronic components therefore reduce the amount of space consumed by sensors, and allow for reduced form factors. Moreover, embodiments provide sensor geometries that further improve and/or provide new sensor functionality. Certain embodiments therefore increase a number of sensor components for new and/or different measurement functions and sensor reliability, improve control of typical optical functions, and provide relatively compact form factors. Embodiments may therefore improve the reliability, functionality, and performance of wearable devices.

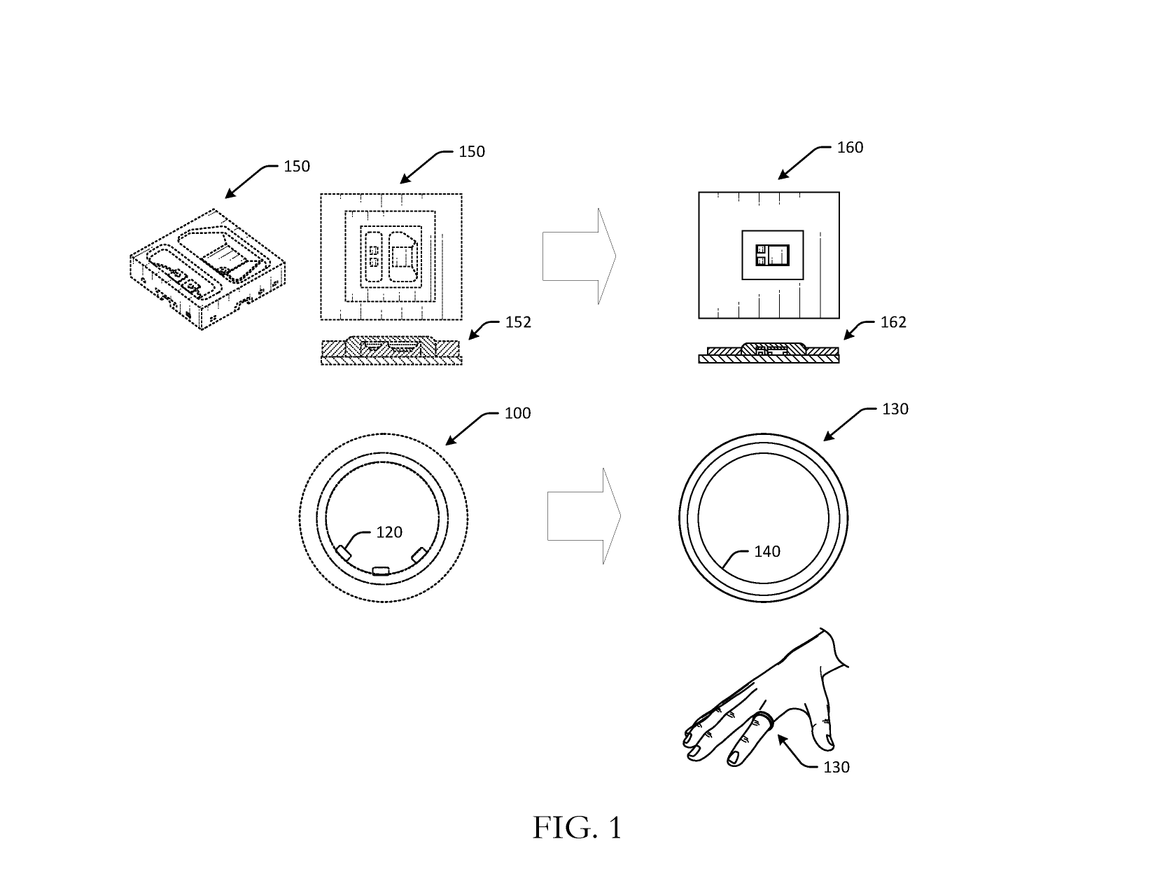

Referring to FIG. 1, an example use case for smart rings having compact form factors is depicted in accordance with one or more embodiments of the disclosure. Although discussed in the context of wearable ring devices, other embodiments may be directed to any suitable use case where wearable devices are used, such as wristwatches, bands, and so forth.

In FIG. 1, a typical smart ring 100 is depicted with a number of dimples or raised portions 120 along an inner housing of the typical smart ring 100. The typical smart ring 100 may include one or more packaged optoelectronic components 150, such as PPG sensor components, light emitting diode components, and the like. The packaged optoelectronic component 150 is depicted in perspective and top views, as well as cross-sectional view 152 to depict a thickness of the packaged optoelectronic component 150. Such size and thickness consumes valuable internal space inside the typical smart ring 100, resulting in a bulky form factor. Moreover, due to space constraints and performance optimization, the raised protrusions 120 may be needed, and may be aesthetically unpleasing and affect comfort of a user wearing the typical smart ring 100.

In contrast, a wearable ring device 130, which may include at least the same functionality and/or sensor components as the typical smart ring 100, in accordance with one or more embodiments is depicted in FIG. 1. As depicted in the example of FIG. 1, a user may wear the wearable ring device 130 on an index finger or other finger. In some embodiments, the wearable ring device 130 may have a smooth inner housing 140, or may have raised protrusions along the inner housing 140 that are reduced in size relative to the raised protrusions 120 of the typical smart ring 100. As depicted in FIG. 1, although the wearable ring device 130 and the typical smart ring 100 have a same outer diameter, an inner diameter of the wearable ring device 130 is greater than the inner diameter of the typical smart ring 100. This is due to a reduced thickness of the wearable ring device 130. As a result, a user wearing the same size ring may be more comfortable due to reduced thickness and reduced or removed raised protrusions. The wearable ring device 130 may include chip-level optoelectronic components 160, such as chip- level light emitting diodes, photodetectors, and/or other components. As depicted in cross- sectional view 162, the chip-level optoelectronic component 160 may have a reduced thickness relative to the packaged optoelectronic component 150. In addition to reducing thickness of the wearable ring device 130, additional and/or different sensors or components can be included in the wearable ring device 130, thereby providing the ability to complete additional measurements previously restricted due to internal space.

Example embodiments of the disclosure provide a number of technical features or technical effects. For example, in accordance with example embodiments of the disclosure, certain embodiments may improve processing speed, sensor measurement, and/or device performance. The above examples of technical features and/or technical effects of example embodiments of the disclosure are merely illustrative and not exhaustive.

One or more illustrative embodiments of the disclosure have been described above. The above-described embodiments are merely illustrative of the scope of this disclosure and are not intended to be limiting in any way. Accordingly, variations, modifications, and equivalents of the embodiments disclosed herein are also within the scope of this disclosure. The above-described embodiments and additional and/or alternative embodiments of the disclosure will be described in detail hereinafter through reference to the accompanying drawings.

### ILLUSTRATIVE EMBODIMENTS AND USE CASES

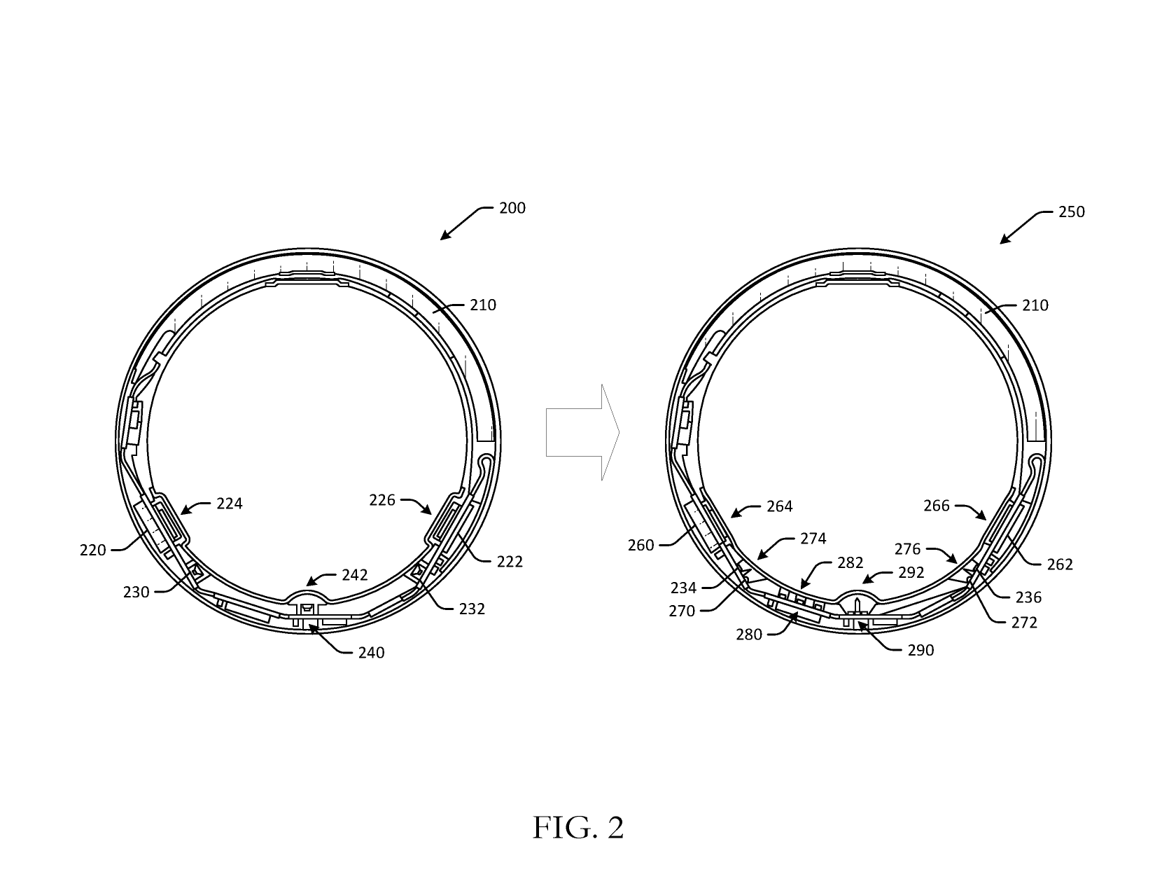

FIG. 2 is a schematic illustration of an example difference in form factor for a wearable ring device having a compact form factor in accordance with one or more embodiments of the disclosure. Other embodiments may include additional or fewer components. The illustration of FIG. 2 may not be to scale, and may not be illustrated to scale with respect to other figures. The wearable devices illustrated in FIG. 2 may be the wearable devices discussed with respect to FIG. 1.

In FIG. 2, a typical smart ring 200 is depicted in a cross-sectional view. The optical structures used to sense one or more measurements of the typical smart ring 200 are limited by the package sizes of the light emitting diode and photodetector assemblies. For example, the typical smart ring 200 may include a first packaged photodetector 220 with a corresponding first raised protrusion 224 on an inner housing of the typical smart ring 200. The typical smart ring 200 may include a second packaged photodetector 222 on another side of the typical smart ring 200, with a corresponding second raised protrusion 226. The typical smart ring 200 may include a first packaged light emitting diode 230, which may be configured to output light having a green color, and a second packaged light emitting diode 232, which may be configured to output light having a green color. The typical smart ring 200 may include a third packaged light emitting diode 240, which may be configured to output infrared light and/or light having a red color, along with a corresponding third raised protrusion 242. The typical smart ring 200 may include a curved battery 210 disposed in an upper half or upper portion of the housing of the typical smart ring 200 ("upper," "lower," etc. are used herein as relational and not absolute terms). The packaged optoelectronic components may be disposed on a printed circuit board in a lower portion of the typical smart ring 200.

A wearable ring device 250 is depicted in the example of FIG. 2 with chip-level optoelectronic components. The wearable ring device 250 may have raised protrusions of reduced size and/or may be devoid of raised protrusions, and may have larger photodetector areas resulting in improved sensor performance, particularly for heart rate or PPG measurements. The wearable ring device 250 may include reflective structures that improve optical sensor performance and may allow for additional light sources and/or other optical sensing functionality. Moreover, optoelectronic light source components of the wearable ring device 250 may emit more light using the same footprint relative to the typical smart ring 200. The wearable ring device 250 may be configured to detect one or more physiological metrics of the user via one or more optoelectronic components, such as light emitting diodes of various colors or wavelengths, photodetectors, and/or other components that can be used to detect physiological metrics. In one example, light emitted from a light emitting diode can be detected using a photodetector, with an amount of light detected being indicative of a particular physiological metric. In particular, light emitting diodes that emit red light and green light may be included. Other embodiments may include additional, fewer, and/or different colors. The wearable ring device 250 may be a finger-worn wearable device, and have a housing having an interior diameter between 12 mm and 24 mm and an exterior diameter between 18 mm and 30 mm for at least a portion of the wearable ring device 250. The wearable ring device 250 may include the curved battery 210 disposed in an upper half or upper portion of the housing of the wearable ring device 250. In some embodiments, the curved battery may be larger in size relative to the typical smart ring 200 due to increased internal space available in the wearable device 250. The optoelectronic components of the wearable ring device 250 may be directly attached to a printed circuit board in a lower portion of the wearable ring device 250. The wearable ring device 250 may include a first photodetector 260 with a corresponding first raised protrusion 264 on an inner housing of the wearable ring device 250.

The wearable ring device 250 may include a second photodetector 262 on another side of the wearable ring device 250, with a corresponding second raised protrusion 266. The wearable ring device 250 may include a first light emitting diode 234, which may be configured to output light having a green color, and a second light emitting diode 236, which may be configured to output light having a green color. The wearable ring device 250 may include a third light emitting diode 270, which may be configured to output light having a yellow color, and a fourth light emitting diode 272, which may also be configured to output light having a yellow color. The wearable ring device 250 may include one or more laser diode assemblies 280 configured to optionally output infrared light. The laser diode assembly 280 may be a vertical cavity surface emitting laser assembly, an edge emitting laser diode assembly, or another type of laser assembly configured to output lights of different wavelengths. The wearable ring device 250 may include a fifth light emitting diode 290, which may be configured to output infrared light and/or light having a red color, along with a corresponding third raised protrusion 292. In some embodiments, the wearable ring device 250 may include a sixth light emitting diode adjacent to fifth light emitting diode 290, which may also be configured to output infrared light and/or light having a red color. In some embodiments, the fifth and sixth light emitting diodes may be part of a single assembly, whereas in other embodiments, the fifth and sixth light emitting diodes may be discrete components. In some embodiments, one or more of the light emitting diodes may be disposed along an upper portion of the wearable ring device 250 (e.g., adjacent to the curved battery 210, etc.). For example, the first light emitting diode may be disposed in the upper half of the housing, and the third light emitting diode may be disposed in the lower half of the housing. This may be due to additional internal space for curved batteries of different geometric configurations. Other light emitting diode and/or optoelectronic component placement configurations may be used.

The optoelectronic components of the wearable ring device 250 may be chip-level optoelectronic components. Accordingly, the optoelectronic components, such as the light emitting diodes, laser diode assemblies, and/or photodetectors, etc., may be coupled directly to the printed circuit board of the wearable ring device 250. As a result, the wearable ring device 250 may have a compact form factor. The raised portions of the wearable ring device 250 may be formed of an optically clear material, and may optionally form a dome or dimple. For example, the raised portion 292 may be disposed such that light output from the third light emitting diode propagates through the dome. Optional materials include glass, epoxy, transparent polymers, etc.

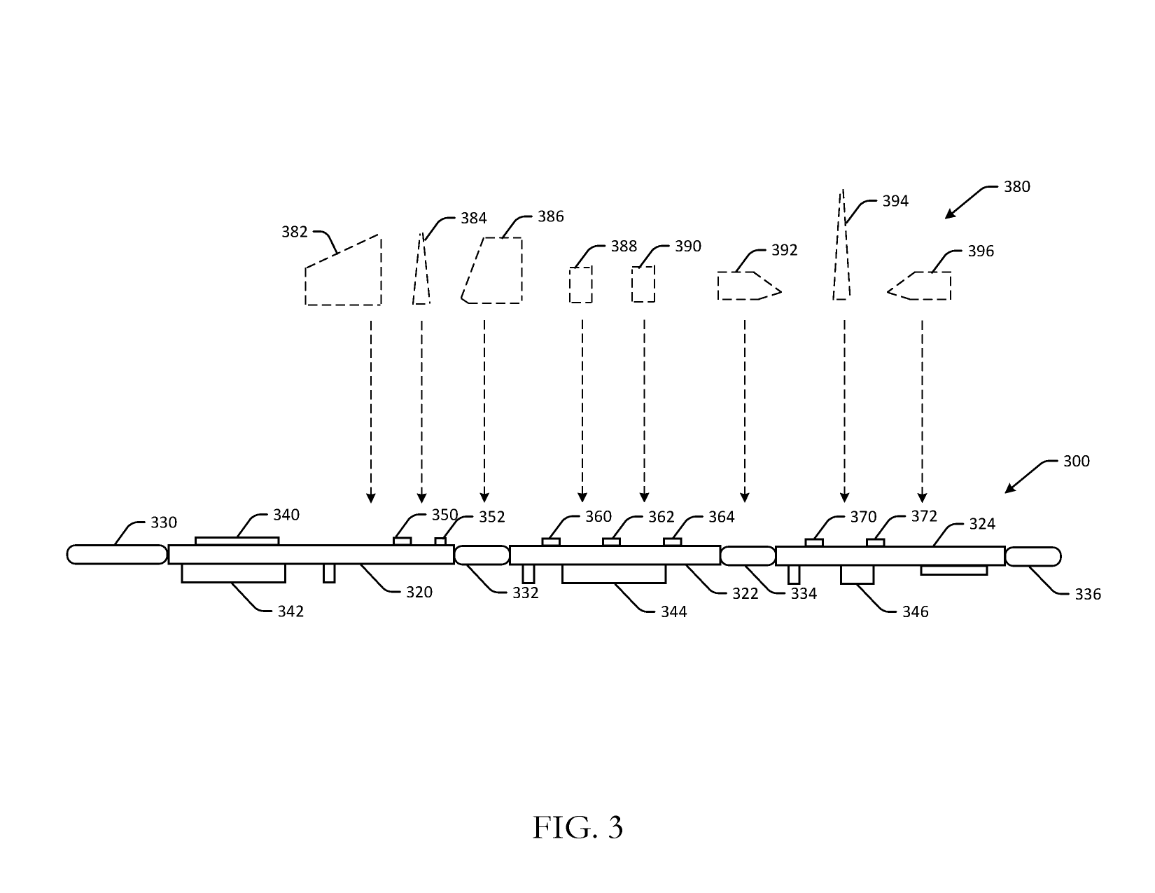

The wearable ring device 250 may include one or more reflective structures that enhance sensor performance and allow for the use of chip-level optoelectronic components. For example, a first reflective structure 274 may be disposed between the first light emitting diode 234 and the third light emitting diode 270, a second reflective structure 282 may be disposed between light emitting diodes of the laser diode assembly 280, a third reflective structure 276 may be disposed between the second light emitting diode 236 and the fourth light emitting diode 272, and so forth. Reflective structures are discussed in more detail at least with respect to FIG. 3.

Accordingly, the wearable ring device 250 may be a finger-worn wearable ring device having a housing having an interior diameter between 12 mm and 24 mm and an exterior diameter between 18 mm and 30 mm for at least a portion of the finger-worn wearable ring device, and may have a compact form factor.

FIG. 3 is a schematic illustration of an example printed circuit board 300 with chip- level optoelectronic components and reflective structures for use with a smart ring having a compact form factor in accordance with one or more embodiments of the disclosure. Other embodiments may include additional or fewer components. The illustration of FIG. 3 is not to scale, and may not be illustrated to scale with respect to other figures. The components illustrated in FIG. 3 may be used with the wearable devices discussed with respect to at least FIGS. 1-2.

In FIG. 3, the printed circuit board 300 is depicted in a flattened orientation and may be the printed circuit board depicted in the wearable ring device 250 of FIG. 2. The printed circuit board 300 may include one or more rigid portions and one or more flexible portions. For example, the printed circuit board 300 may include a first rigid portion 320, a second rigid portion 322, a third rigid portion 324, and so forth. One or more flexible portions may separate the respective rigid portions. For example, a first flexible portion 332 may separate the second rigid portion 322 from the first rigid portion 320, and a second flexible portion 334 may separate the third rigid portion 324 from the second rigid portion 322. Additional flexible portions, such as a third flexible portion 330 and/or a fourth flexible portion 336 may be included.

One or more chip-level optoelectronic components may be directly attached or coupled to the printed circuit board 300. For example, a first light emitting chip, such as a first light emitting diode 350 may be disposed on the first rigid portion 320 of the printed circuit board 300. The first light emitting diode 350 may be configured to output green light. A second light emitting diode 352 may be disposed on the first rigid portion 320 of the printed circuit board 300, where the second light emitting diode 352 may be configured to output yellow light. A third light emitting diode 370 may be disposed on the third rigid portion 324 of the printed circuit board 300, where the third light emitting diode 370 may be configured to output red light. The first light emitting diode 350, the second light emitting diode 352, and the third light emitting diode 370 may be chip-level optoelectronic components, and may therefore be directly attached to the printed circuit board 300.

Embodiments may include additional optoelectronic components, such as an optional fourth light emitting diode 372 disposed on the printed circuit board 300 adjacent to the third light emitting diode 300, where the fourth light emitting diode 372 may be configured to output red light. One or more light emitting diodes that may be part of a laser diode assembly 344, such as a fifth light emitting diode 360, a sixth light emitting diode 362, and a seventh light emitting diode 364, may be disposed on the second rigid portion 322 of the printed circuit board 300. Such light emitting diodes may be configured to output light having a red color, infrared light, and/or other wavelengths of light. A first photodetector chip 342 and corresponding photodetector component 340 may be disposed on the first rigid portion 320. Any number of photodetectors may be included. The photodetector component 340 may have a larger surface area, thereby improving sensor performance, relative to typical designs due to increased available footprint on the printed circuit board 300. The photodetector component 340 may be a first photodiode disposed on the first rigid portion 320 of the printed circuit board 300, and may be configured to detect light output from at least one of the first light emitting diode 350, the second light emitting diode 352, or the third light emitting diode 370.

One or more reflective structures 380 may be disposed on the printed circuit board 300. For example, a first reflective structure 394 may be disposed on the printed circuit board 300 between the third light emitting diode 370 and the fourth light emitting diode 372. A second reflective structure 384 may be disposed on the printed circuit board 300 between the first light emitting diode 350 and the second light emitting diode 352. In some embodiments, both the first reflective structure 394 and the second reflective structure 384 have a substantially triangular cross-sectional geometry, and may optionally be formed at least partially of metal. The first and second reflective structures may have the same dimensions and may act to reflect light emitted from adjacent optoelectronic light components in a particular manner.

Additional reflective structures may be included and may also be formed at least partially of metal. For example, the second reflective structure 384 may be disposed on a first side of the second light emitting diode 352, and a third reflective structure 386 may be disposed on the printed circuit board 300 on a second side of the second light emitting diode 352, as depicted in the example of FIG. 3. The third reflective structure 386 may have a different configuration than the second reflective structure 384. For example, the third reflective structure 386 may not have a triangular cross-sectional geometry. A fourth reflective structure 382 may be disposed adjacent to the first light emitting diode 350 and may have a trapezoidal or other geometric configuration. A fifth reflective structure 388 may be disposed between the fifth light emitting diode 360 and the sixth light emitting diode 362. A sixth reflective structure 390, which may have the same configuration as the fifth reflective structure 388, may be disposed between the sixth light emitting diode 362 and the seventh light emitting diode 364. A seventh reflective structure 392 may be disposed on an opposite side of the third light emitting diode 370 relative to the first reflective structure 394, and may be adjacent to the second flexible portion 334 of the printed circuit board 300. An eighth reflective structure 396 may be disposed adjacent to the fourth light emitting diode 372. The individual reflective structure configurations, including geometry and dimensions, may be optimized to reflect light in a particular manner, so as to address the lack of integrated package reflective structures.

Accordingly, in one embodiment, a wearable device may include a printed circuit board having a first portion, a second portion, and a third portion, where the second portion is disposed between the first portion and the third portion. The device may include a first light emitting chip disposed on the first portion of the printed circuit board, a second light emitting chip disposed on the first portion of the printed circuit board, and at least one of a laser diode assembly configured to output infrared light, or a vertical cavity surface emitting laser assembly. The at least one of the laser diode assembly or the vertical cavity surface emitting laser assembly may be disposed on the second portion of the printed circuit board. The device may include a first photodiode disposed on the first portion of the printed circuit board, where the photodiode is configured to detect light output from at least one of the first light emitting chip or the second light emitting chip. The first light emitting chip and the second light emitting chip can be directly attached to the printed circuit board. In some instances, the printed circuit board may include a first flexible portion separating the second portion from the first portion, and a second flexible portion separating the third portion from the second portion. The wearable device may include a third light emitting chip disposed on the third portion of the printed circuit board, a fourth light emitting chip disposed adjacent to the third light emitting chip, a first reflective structure disposed between the third light emitting chip and the fourth light emitting chip, and a second reflective structure disposed between the first light emitting chip and the second light emitting chip. In one example, both the first reflective structure and the second reflective structure have a substantially triangular cross-sectional geometry, and both the first reflective structure and the second reflective structure are formed at least partially of metal.

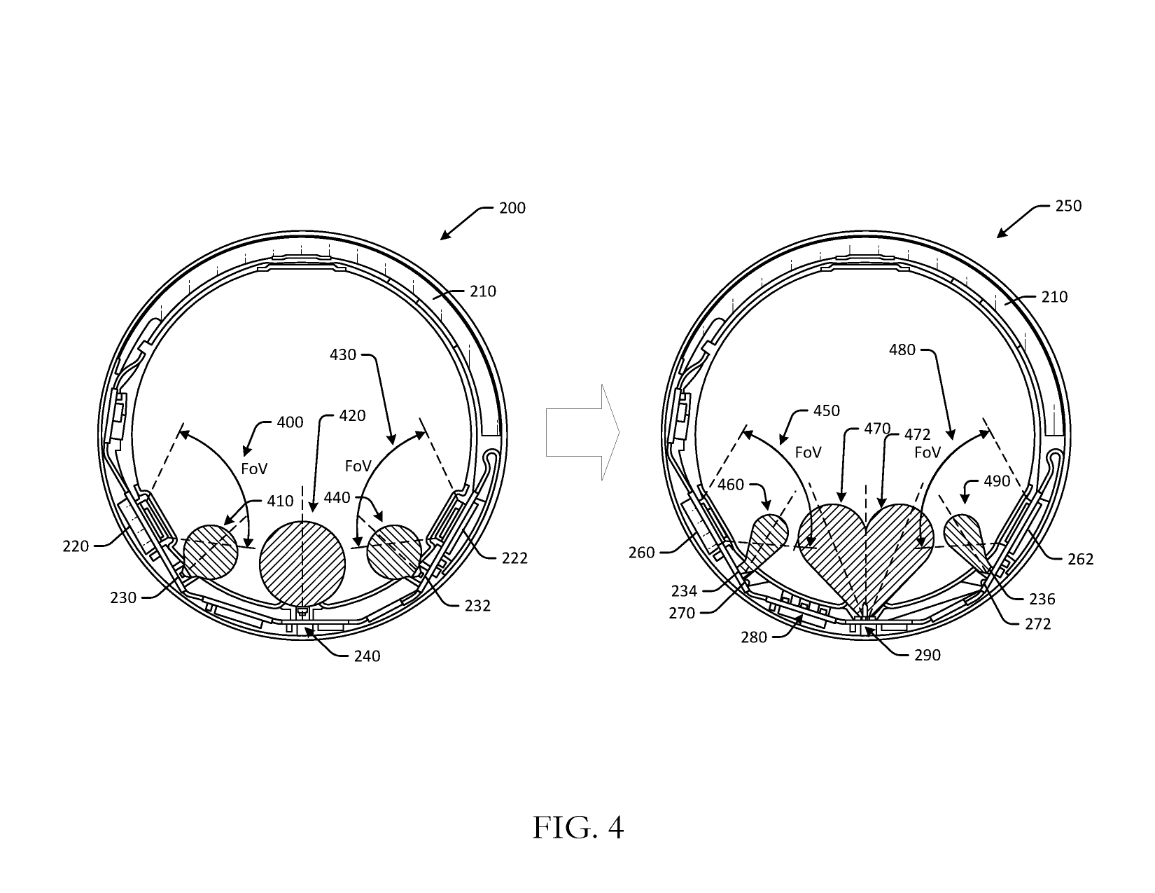

FIG. 4 depicts an example improved sensor function for smart rings having compact form factors relative to a typical wearable device in accordance with one or more embodiments of the disclosure. Other embodiments may include additional or fewer components. The illustration of FIG. 4 is not to scale, and may not be illustrated to scale with respect to other figures. The components illustrated in FIG. 4 may be the same as those discussed with respect to at least FIGS. 1-3.

In FIG. 4, the typical smart ring 200 of FIG. 2 is depicted with example light emission patterns and corresponding fields of view for the respective photodetectors. As depicted in the example of FIG. 4, packaged light sources have predetermined light emission patterns due to the pre-constructed packaging. For example, the first packaged light emitting diode 230, which may be configured to output light having a green color, may have a light emission pattern 410, where emitted light can be detected by at least the first photodetector 220 via a first field of view 400. The second packaged light emitting diode 232, which may be configured to output light having a green color, may have a light emission pattern 440, where emitted light can be detected by at least the second photodetector 222 via a second field of view 430. Light emitted by the third packaged light emitting diode 240, which may be configured to output infrared light and/or light having a red color, may be emitted in a light emission pattern 420 and may be detected by one or both the first photodetector 220 or the second photodetector 222.

In contrast, the wearable ring device 250 may include reflective structures configured to customize light emission patterns for chip-level light source components. For example, the first photodetector 260 may have a first field of view 450, and the second photodetector 262 may have a second field of view 480. The fields of the view and/or light sensitive areas of the photodetectors in the wearable ring device 250 may be relatively larger than the fields of view of the photodetectors of the typical smart ring 200.

Light emission patterns can be customized to improve performance of sensor function, as well as to allow for additional sensing features. For example, light emitted by the first light emitting diode 234, which may be configured to output light having a green color, may have a first light emission pattern 460, and light emitted by the second light emitting diode 236, which may be configured to output light having a green color, may have a second light emission pattern 490. Light emitted by the fifth light emitting diode 290, which may be configured to output infrared light and/or light having a red color, may have a third light emission pattern 470 and/or fourth light emission pattern 472. As depicted in the example of FIG. 4, the light emission patterns are targeted and can be enhanced and/or directed in a desired direction as a result of the reflective structures, unlike the light emission patterns of the typical smart ring 200. In addition, light direction can be modified to increase an amount of light reflected and/or directed to the respective photodetectors. Such improvements may be completed without increasing an amount of light emitted and/or power consumed by the wearable ring device 250.

As a result, the wearable ring device 250 may have improved optical structures, increased optical functionality, increased control over light emission patterns, and increased light sensitive areas (e.g., increase photodetector surface area, etc.) to collect increased amounts of light. Sensor functionality related to hydration, hemoglobin, lipid count, ethanol, glucose, and/or other biometrics may be facilitated in a compact form factor due to the inclusion of laser diode assemblies.

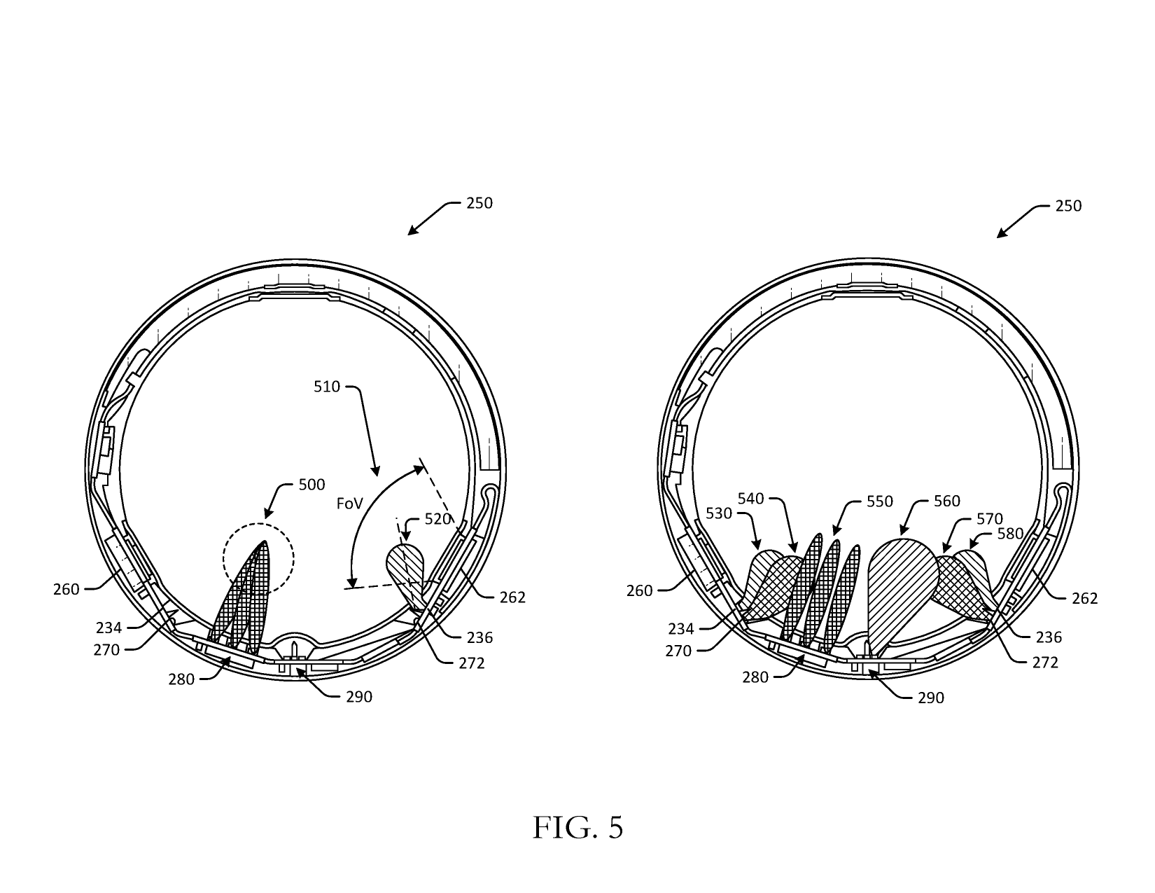

FIG. 5 is a schematic illustration of an additional example improved sensor function for smart rings having compact form factors in accordance with one or more example embodiments of the disclosure. Other embodiments may include additional or fewer components. The illustration of FIG. 5 is not to scale, and may not be illustrated to scale with respect to other figures. The components illustrated in FIG. 5 may be the same as those discussed with respect to at least FIGS. 1-4.

In FIG. 5, the wearable ring device 250 is depicted with an example light emission pattern 500 for the laser diode assembly 280, which may be a vertical cavity surface emitting laser assembly, an edge emitting laser diode assembly, or another type of laser assembly configured to output lights of different wavelengths. The light output may be arranged to merge at a predetermined point. In addition to, or instead of, reflective structures, embodiments may include optical microstructures integrated on a lower surface (e.g., an inner surface, etc.) of a glass window on an inner housing of the wearable ring device 250 through with the light propagates as it is emitted. Other embodiments may use different types of materials for the optically clear window. Examples of optical microstructures include dents, raised protrusions (e.g., circular protrusions, rectangular protrusions, etc.), stippling, and so forth. Optical microstructures may be formed in various patterns along the inner-facing surface of the window to direct light in a certain direction. Such emitted light may be detected via a field of view 510 of the second photodetector 262. The light emitted by second light emitting diode 236, which may be configured to output light having a green color, may have a light emission pattern 520 that is also detected at least partially by the second photodetector 262.

The wearable ring device 250 may include additional light sources to increase reliability and/or provide additional measurements. For example, the light emitted by the first light emitting diode 234, which may be configured to output light having a green color, may have a first light emission pattern 530, and light emitted by the second light emitting diode 236, which may be configured to output light having a green color, may have a second light emission pattern 280. Light emitted by the fifth light emitting diode 290, which may be configured to output infrared light and/or light having a red color, may have a third light emission pattern 560. Light emitted by the third light emitting diode 270, which may be configured to output light having a yellow color, may have a fourth light emission pattern 540, and light emitted by the fourth light emitting diode 272, which may also be configured to output light having a yellow color, may have a fifth light emission pattern 570. In the right-side example of FIG. 5, a sixth light emission pattern 550 for the laser diode assembly 280, which may be a vertical cavity surface emitting laser assembly, an edge emitting laser diode assembly, or another type of laser assembly configured to output lights of different wavelengths, may be different than the merging light emission pattern 500 on the left-side example. Such modifications may be due to optical microstructure configuration and/or reflective structure arrangement.

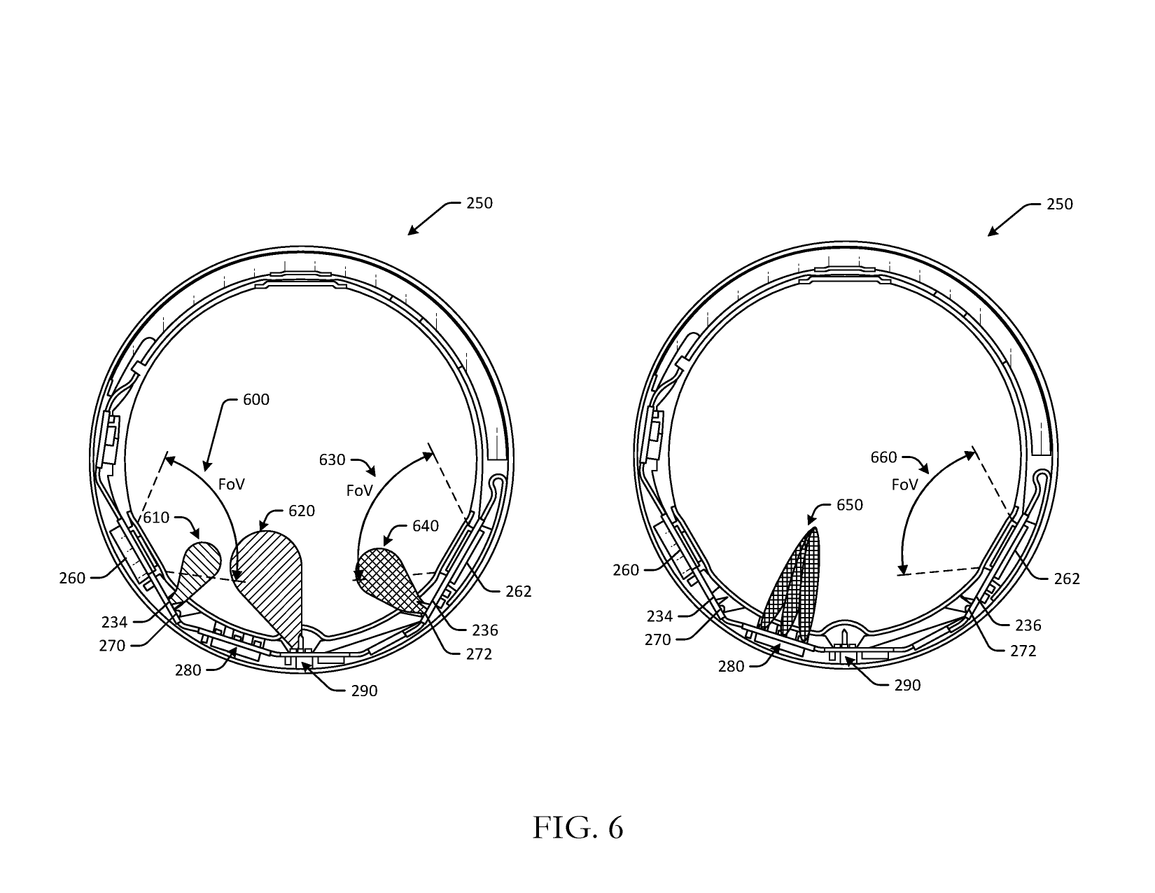

FIG. 6 is a schematic illustration of an additional example improved sensor function for smart rings having compact form factors in accordance with one or more example embodiments of the disclosure. Other embodiments may include additional or fewer components. The illustrations of FIG. 6 are not to scale, and may not be illustrated to scale with respect to other figures. The components illustrated in FIG. 6 may be the same as those discussed with respect to at least FIGS. 1-5.

In FIG. 6, the wearable ring device 250 is depicted with an example light emission pattern for directional hemispherical reflectance and/or related measurement types using green, yellow, and red colored lights. In a left-side example of FIG. 6, light emitted by the first light emitting diode 234, which may be configured to output light having a green color, may have a first light emission pattern 610, and light emitted by the second light emitting diode 236, which may be configured to output light having a green color, may have a second light emission pattern 640. Light emitted by the fifth light emitting diode 290, which may be configured to output infrared light and/or light having a red color, may have a third light emission pattern 620. The first photodetector 260 may have a first field of view 600 and the second photodetector 262 may have a second field of view 630 to collect emitted and/or reflected light.

In a right-side example of FIG. 6, the second photodetector 262 may have a field of view 660, which may collect light emitted by and/or reflected light from the laser diode assembly 280. The laser diode assembly 280 may have a light emission pattern 650 that can be optimized in direction for collection by the first photodetector 260 and/or the second photodetector 262. The light emission pattern depicted in the right-side example of FIG. 6 may be used for spectral measurement of a skin hydration level in one instance.

One or more operations of the methods, process flows, or use cases of FIGS. 1-6 may have been described above as being performed by a user device, or more specifically, by one or more program module(s), applications, or the like executing on a device. It should be appreciated, however, that any of the operations of the methods, process flows, or use cases of FIGS. 1-6 may be performed, at least in part, in a distributed manner by one or more other devices, or more specifically, by one or more program module(s), applications, or the like executing on such devices. In addition, it should be appreciated that processing performed in response to the execution of computer-executable instructions provided as part of an application, program module, or the like may be interchangeably described herein as being performed by the application or the program module itself or by a device on which the application, program module, or the like is executing. While the operations of the methods, process flows, or use cases of FIGS. 1-6 may be described in the context of the illustrative devices, it should be appreciated that such operations may be implemented in connection with numerous other device configurations.

Although specific embodiments of the disclosure have been described, one of ordinary skill in the art will recognize that numerous other modifications and alternative embodiments are within the scope of the disclosure. For example, any of the functionality and/or processing capabilities described with respect to a particular device or component may be performed by any other device or component. Further, while various illustrative implementations and architectures have been described in accordance with embodiments of the disclosure, one of ordinary skill in the art will appreciate that numerous other modifications to the illustrative implementations and architectures described herein are also within the scope of this disclosure.

Certain aspects of the disclosure are described above with reference to block and flow diagrams of systems, methods, apparatuses, and/or computer program products according to example embodiments. It will be understood that one or more blocks of the block diagrams and flow diagrams, and combinations of blocks in the block diagrams and the flow diagrams, respectively, may be implemented by the execution of computer-executable program instructions. Likewise, some blocks of the block diagrams and flow diagrams may not necessarily need to be performed in the order presented, or may not necessarily need to be performed at all, according to some embodiments. Further, additional components and/or operations beyond those depicted in blocks of the block and/or flow diagrams may be present in certain embodiments.

Accordingly, blocks of the block diagrams and flow diagrams support combinations of means for performing the specified functions, combinations of elements or steps for performing the specified functions, and program instruction means for performing the specified functions. It will also be understood that each block of the block diagrams and flow diagrams, and combinations of blocks in the block diagrams and flow diagrams, may be implemented by special-purpose, hardware-based computer systems that perform the specified functions, elements or steps, or combinations of special-purpose hardware and computer instructions.

### ILLUSTRATIVE COMPUTER ARCHITECTURE

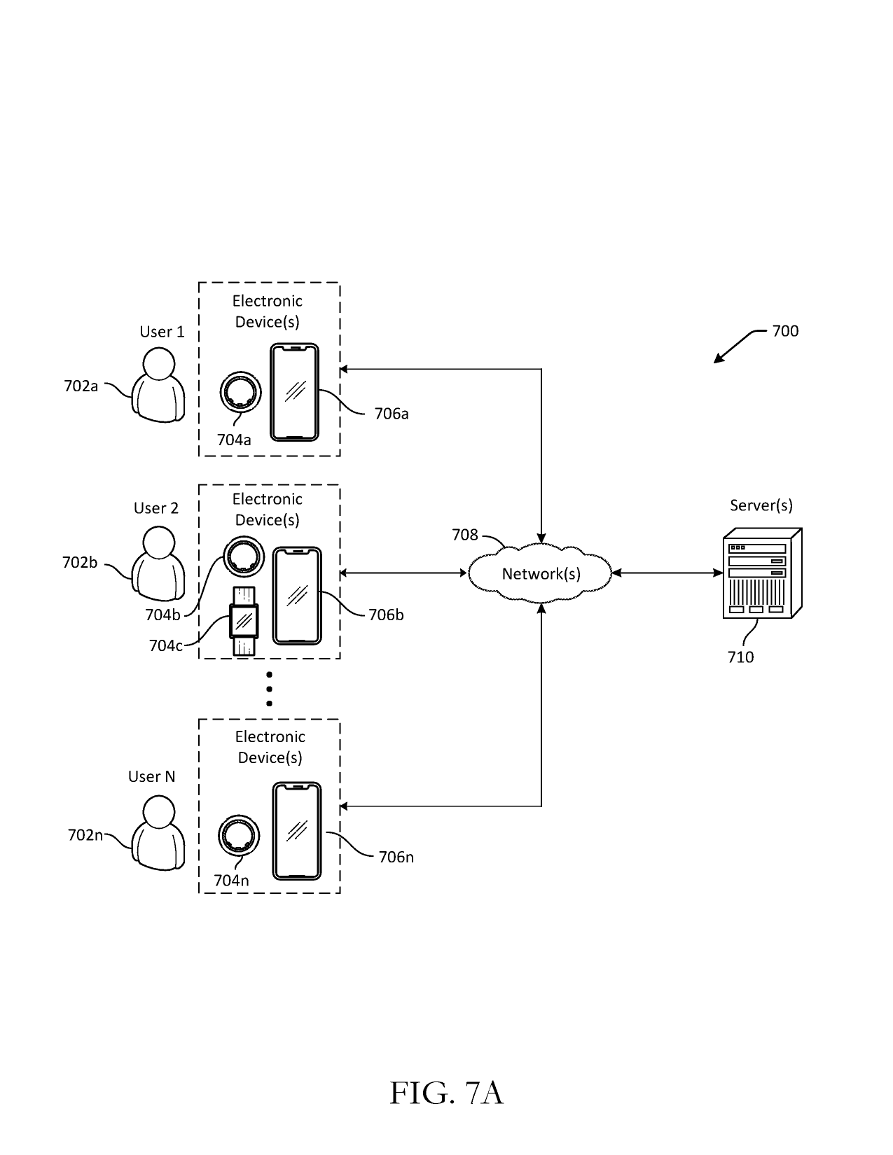

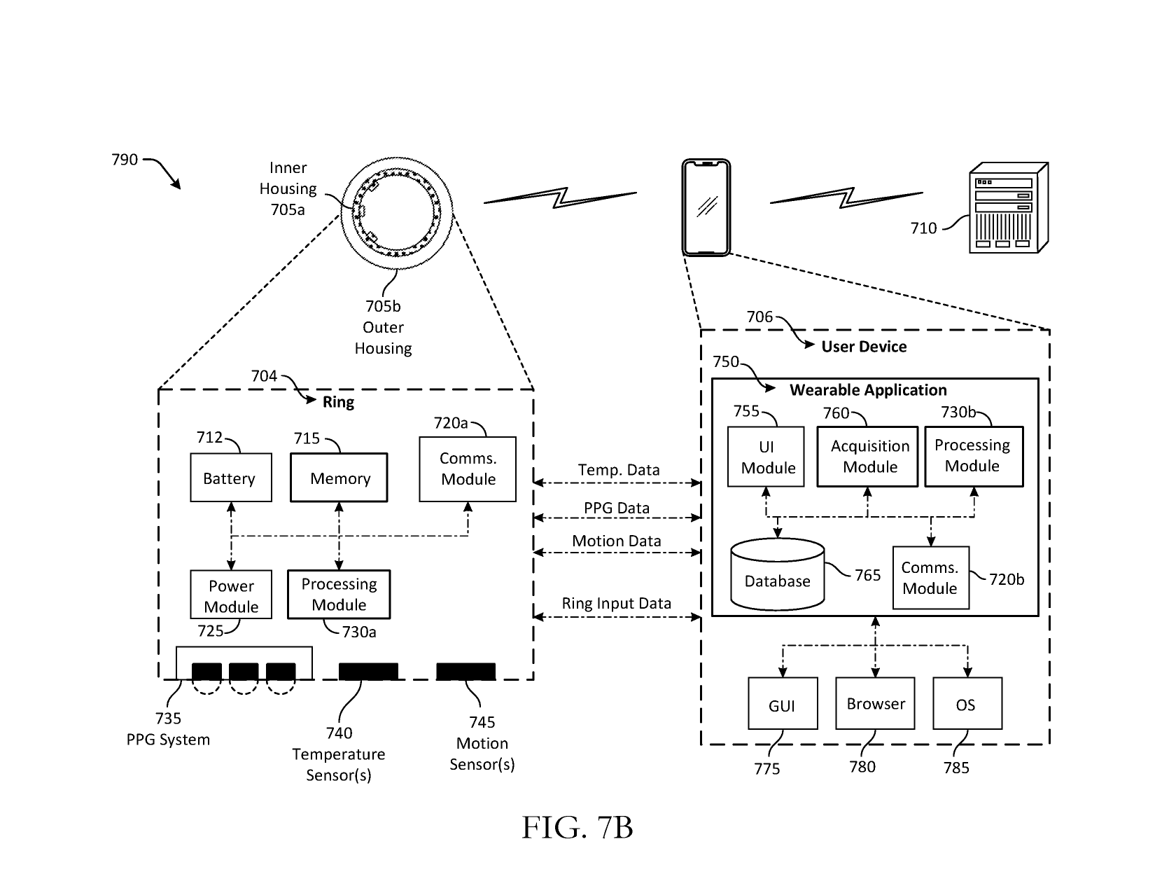

FIGS. 7A-7B schematically illustrate an example of a system 700 that supports techniques for active and passive companion ring detection for wearable devices in accordance with aspects of the present disclosure. The system 700 includes a plurality of electronic devices (e.g., wearable devices 704, user devices 706) that may be worn and/or operated by one or more users 702. The system 700 further includes a network 708 and one or more servers 710.

The electronic devices may include any electronic devices known in the art, including wearable devices 704 (e.g., ring wearable devices, watch wearable devices, etc.), user devices 706 (e.g., smartphones, laptops, tablets), and the like. The electronic devices associated with the respective users 702 may include one or more of the following functionalities: 1) measuring physiological data, 2) storing the measured data, 3) processing the data, 4) providing outputs (e.g., via GUIs and/or wirelessly coupled displays, etc.) to a user 702 based on the processed data, and 5) communicating data with one another and/or other computing devices. Different electronic devices may perform one or more of the functionalities.

Example wearable devices 704 may include wearable computing devices, such as a ring computing device (hereinafter "ring") configured to be worn on a user's 702 finger, a wrist computing device (e.g., a smart watch, fitness band, or bracelet) configured to be worn on a user's 702 wrist, and/or a head mounted computing device (e.g., glasses/goggles). Wearable devices 704 may also include bands, straps (e.g., flexible or inflexible bands or straps), stick-on sensors, and the like, that may be positioned in other locations, such as bands around the head (e.g., a forehead headband), arm (e.g., a forearm band and/or bicep band), and/or leg (e.g., a thigh or calf band), behind the ear, under the armpit, and the like. Wearable devices 704 may also be attached to, or included in, articles of clothing. For example, wearable devices 704 may be included in pockets and/or pouches on clothing. As another example, wearable device 704 may be clipped and/or pinned to clothing, or may otherwise be maintained within the vicinity of the user 702. Example articles of clothing may include, but are not limited to, hats, shirts, gloves, pants, socks, outerwear (e.g., jackets), and undergarments. In some implementations, wearable devices 704 may be included with other types of devices such as training/sporting devices that are used during physical activity. For example, wearable devices 704 may be attached to, or included in, a bicycle, skis, a tennis racket, a golf club, and/or training weights.

Much of the present disclosure may be described in the context of a ring wearable device 704. Accordingly, the terms "ring 704," "wearable device 704," and like terms, may be used interchangeably, unless noted otherwise herein. However, the use of the term "ring 704" is not to be regarded as limiting, as it is contemplated herein that aspects of the present disclosure may be performed using other wearable devices (e.g., watch wearable devices, necklace wearable device, bracelet wearable devices, earring wearable devices, anklet wearable devices, and the like).

In some aspects, user devices 706 may include handheld mobile computing devices, such as smartphones and tablet computing devices. User devices 706 may also include personal computers, such as laptop and desktop computing devices. Other example user devices 706 may include server computing devices that may communicate with other electronic devices (e.g., via wireless communication, via the Internet, etc.). In some implementations, computing devices may include medical devices, such as external wearable computing devices (e.g., Holter monitors). Medical devices may also include implantable medical devices, such as pacemakers and cardioverter defibrillators. Other example user devices 706 may include home computing devices, such as internet of things (IoT) devices (e.g., IoT devices), smart televisions, smart speakers, smart displays (e.g., video call displays), hubs (e.g., wireless communication hubs), security systems, smart appliances (e.g., thermostats and refrigerators), and fitness equipment.

Some electronic devices (e.g., wearable devices 704, user devices 706) may measure physiological parameters of respective users 702, such as photoplethysmography waveforms, continuous skin temperature, a pulse waveform, respiration rate, heart rate, heart rate variability (HRV), actigraphy, galvanic skin response, pulse oximetry, blood oxygen saturation (SpO2), blood sugar levels (e.g., glucose metrics), and/or other physiological parameters. Some electronic devices that measure physiological parameters may also perform some or all of the calculations or determinations described herein. Some electronic devices may not measure physiological parameters, but may perform some or all of the calculations or determinations described herein. For example, a ring (e.g., wearable device 704), mobile device application, or a server computing device may process received physiological data that was measured by other devices.

In some implementations, a user 702 may operate, or may be associated with, multiple electronic devices, some of which may measure physiological parameters and some of which may process the measured physiological parameters. In some implementations, a user 702 may have a ring (e.g., wearable device 704) that measures physiological parameters. The user 702 may also have, or be associated with, a user device 706 (e.g., mobile device, smartphone), where the wearable device 704 and the user device 706 are communicatively coupled to one another. In some cases, the user device 706 may receive data from the wearable device 704 and perform some or all of the calculations or determinations described herein. In some implementations, the user device 706 may also measure physiological parameters described herein, such as motion and/or activity parameters.

For example, as illustrated in FIG. 7A, a first user 702-a (User 1) may operate, or may be associated with, a wearable device 704-a (e.g., ring 704-a) and a user device 706-a that may operate as described herein. In this example, the user device 706-a associated with user 702-a may process and/or store physiological parameters measured by the ring 704-a. Comparatively, a second user 702-b (User 2) may be associated with a ring 704-b, a watch wearable device 704-c (e.g., watch 704-c), and a user device 706-b, where the user device 706-b associated with user 702-b may process and/or store physiological parameters measured by the ring 704-b and/or the watch 704-c. Moreover, an nth user 702-n (User N) may be associated with an arrangement of electronic devices described herein (e.g., ring 704-n, user device 706-n). In some aspects, wearable devices 704 (e.g., rings 704, watches 704) and other electronic devices may be communicatively coupled to the user devices 706 of the respective users 702 via Bluetooth, Wi- Fi, and other wireless protocols. Moreover, in some cases, the wearable device 704 and the user device 706 may be included within (or make up) the same device. For example, in some cases, the wearable device 704 may be configured to execute an application associated with the wearable device 704, and may be configured to display data via a GUI.

In some implementations, the rings 704 (e.g., wearable devices 704) of the system 700 may be configured to collect physiological data from the respective users 702 based on arterial blood flow within the user's finger. In particular, a ring 704 may utilize one or more light- emitting components, such as LEDs (e.g., red LEDs, green LEDs) that emit light on the palm- side of a user's finger to collect physiological data based on arterial blood flow within the user's finger. In general, the terms light-emitting components, light-emitting elements, and like terms, may include, but are not limited to, LEDs, micro LEDs, mini LEDs, laser diodes (LDs) (e.g., vertical cavity surface-emitting lasers (VCSELs), and the like.

In some cases, the system 700 may be configured to collect physiological data from the respective users 702 based on blood flow diffused into a microvascular bed of skin with capillaries and arterioles. For example, the system 700 may collect photoplethysmogram (PPG) data based on a measured amount of blood diffused into the microvascular system of capillaries and arterioles. In some implementations, the ring 704 may acquire the physiological data using a combination of both green and red LEDs. The physiological data may include any physiological data known in the art including, but not limited to, temperature data, accelerometer data (e.g., movement/motion data), heart rate data, HRV data, blood oxygen level data, or any combination thereof.

The use of both green and red LEDs may provide several advantages over other solutions, as red and green LEDs have been found to have their own distinct advantages when acquiring physiological data under different conditions (e.g., light/dark, active/inactive) and via different parts of the body, and the like. For example, green LEDs have been found to exhibit better performance during exercise. Moreover, using multiple LEDs (e.g., green and red LEDs) distributed around the ring 704 has been found to exhibit superior performance as compared to wearable devices that utilize LEDs that are positioned close to one another, such as within a watch wearable device. Furthermore, the blood vessels in the finger (e.g., arteries, capillaries) are more accessible via LEDs as compared to blood vessels in the wrist. In particular, arteries in the wrist are positioned on the bottom of the wrist (e.g., palm-side of the wrist), meaning only capillaries are accessible on the top of the wrist (e.g., back of hand side of the wrist), where wearable watch devices and similar devices are typically worn. As such, utilizing LEDs and other sensors within a ring 704 has been found to exhibit superior performance as compared to wearable devices worn on the wrist, as the ring 704 may have greater access to arteries (as compared to capillaries), thereby resulting in stronger signals and more valuable physiological data.

The electronic devices of the system 700 (e.g., user devices 706, wearable devices 704) may be communicatively coupled to one or more servers 710 via wired or wireless communication protocols. For example, as shown in FIG. 7A, the electronic devices (e.g., user devices 706) may be communicatively coupled to one or more servers 710 via a network 708. The network 708 may implement transfer control protocol and internet protocol (TCP/IP), such as the Internet, or may implement other network 708 protocols. Network connections between the network 708 and the respective electronic devices may facilitate transport of data via email, web, text messages, mail, or any other appropriate form of interaction within a computer network 708. For example, in some implementations, the ring 704-a associated with the first user 702-a may be communicatively coupled to the user device 706-a, where the user device 706-a is communicatively coupled to the servers 710 via the network 708. In additional or alternative cases, wearable devices 704 (e.g., rings 704, watches 704) may be directly communicatively coupled to the network 708.

The system 700 may offer an on-demand database service between the user devices 706 and the one or more servers 710. In some cases, the servers 710 may receive data from the user devices 706 via the network 708, and may store and analyze the data. Similarly, the servers 710 may provide data to the user devices 706 via the network 708. In some cases, the servers 710 may be located at one or more data centers. The servers 710 may be used for data storage, management, and processing. In some implementations, the servers 710 may provide a web- based interface to the user device 706 via web browsers.

In some aspects, the system 700 may detect periods of time that a user 702 is asleep, and classify periods of time that the user 702 is asleep into one or more sleep stages (e.g., sleep stage classification). For example, as shown in FIG. 7A, User 702-a may be associated with a wearable device 704-a (e.g., ring 704-a) and a user device 706-a. In this example, the ring 704-a may collect physiological data associated with the user 702-a, including temperature, heart rate, HRV, respiratory rate, and the like. In some aspects, data collected by the ring 704-a may be input to a machine learning classifier, where the machine learning classifier is configured to determine periods of time that the user 702-a is (or was) asleep. Moreover, the machine learning classifier may be configured to classify periods of time into different sleep stages, including an awake sleep stage, a rapid eye movement (REM) sleep stage, a light sleep stage (non-REM (NREM)), and a deep sleep stage (NREM). In some aspects, the classified sleep stages may be displayed to the user 702-a via a GUI of the user device 706-a. Sleep stage classification may be used to provide feedback to a user 702-a regarding the user's sleeping patterns, such as recommended bedtimes, recommended wake-up times, and the like. Moreover, in some implementations, sleep stage classification techniques described herein may be used to calculate scores for the respective user, such as Sleep Scores, Readiness Scores, and the like.

In some aspects, the system 700 may utilize circadian rhythm-derived features to further improve physiological data collection, data processing procedures, and other techniques described herein. The term circadian rhythm may refer to a natural, internal process that regulates an individual's sleep-wake cycle, that repeats approximately every 24 hours. In this regard, techniques described herein may utilize circadian rhythm adjustment models to improve physiological data collection, analysis, and data processing. For example, a circadian rhythm adjustment model may be input into a machine learning classifier along with physiological data collected from the user 702-a via the wearable device 704-a. In this example, the circadian rhythm adjustment model may be configured to "weight," or adjust, physiological data collected throughout a user's natural, approximately 24-hour circadian rhythm. In some implementations, the system may initially start with a "baseline" circadian rhythm adjustment model, and may modify the baseline model using physiological data collected from each user 702 to generate tailored, individualized circadian rhythm adjustment models that are specific to each respective user 702.

In some aspects, the system 700 may utilize other biological rhythms to further improve physiological data collection, analysis, and processing by phase of these other rhythms. For example, if a weekly rhythm is detected within an individual's baseline data, then the model may be configured to adjust "weights" of data by day of the week. Biological rhythms that may require adjustment to the model by this method include: 1) ultradian (faster than a day rhythms, including sleep cycles in a sleep state, and oscillations from less than an hour to several hours periodicity in the measured physiological variables during wake state; 2) circadian rhythms; 3) non-endogenous daily rhythms shown to be imposed on top of circadian rhythms, as in work schedules; 4) weekly rhythms, or other artificial time periodicities exogenously imposed (e.g. in a hypothetical culture with 12 day "weeks," 12 day rhythms could be used); 5) multi-day ovarian rhythms in women and spermatogenesis rhythms in men; 6) lunar rhythms (relevant for individuals living with low or no artificial lights); and 7) seasonal rhythms.

The biological rhythms are not always stationary rhythms. For example, many women experience variability in ovarian cycle length across cycles, and ultradian rhythms are not expected to occur at exactly the same time or periodicity across days even within a user. As such, signal processing techniques sufficient to quantify the frequency composition while preserving temporal resolution of these rhythms in physiological data may be used to improve detection ofthese rhythms, to assign phase of each rhythm to each moment in time measured, and to thereby modify adjustment models and comparisons of time intervals. The biological rhythm-adjustment models and parameters can be added in linear or non-linear combinations as appropriate to more accurately capture the dynamic physiological baselines of an individual or group of individuals.

In some aspects, the respective devices of the system 700 may support techniques for active and passive companion ring detection for wearable devices. For example, certain calibration curves may be selected based on detection of companion ring material properties, so as to minimize impact to sensor data and related measurements.

It should be appreciated by a person skilled in the art that one or more aspects of the disclosure may be implemented in a system 700 to additionally or alternatively solve other problems than those described above. Furthermore, aspects of the disclosure may provide technical improvements to "conventional" systems or processes as described herein. However, the description and appended drawings only include example technical improvements resulting from implementing aspects of the disclosure, and accordingly do not represent all of the technical improvements provided within the scope of the claims.

FIG. 7B illustrates an example of a system 790 that supports techniques for active and passive companion ring detection for wearable devices in accordance with aspects of the present disclosure. The system 790 may implement, or be implemented by, system 700. In particular, system 790 illustrates an example of a ring 704 (e.g., wearable device 704), a user device 706, and a server 710, as described with reference to FIG. 7A.

In some aspects, the ring 704 may be configured to be worn around a user's finger, and may determine one or more user physiological parameters when worn around the user's finger. Example measurements and determinations may include, but are not limited to, user skin temperature, pulse waveforms, respiratory rate, heart rate, HRV, blood oxygen levels (SpO2), blood sugar levels (e.g., glucose metrics), and the like.

The system 790 further includes a user device 706 (e.g., a smartphone) in communication with the ring 704. For example, the ring 704 may be in wireless and/or wired communication with the user device 706. In some implementations, the ring 704 may send measured and processed data (e.g., temperature data, PPG data, motion/accelerometer data, ring input data, and the like) to the user device 706. The user device 706 may also send data to the ring 704, such as ring 704 firmware/configuration updates. The user device 706 may process data. In some implementations, the user device 706 may transmit data to the server 710 for processing and/or storage.

The ring 704 may include a housing 705 that may include an inner housing 705-a and an outer housing 705-b. In some aspects, the housing 705 of the ring 704 may store or otherwise include various components of the ring including, but not limited to, device electronics, a power source (e.g., battery 712, and/or capacitor), one or more substrates (e.g., printable circuit boards) that interconnect the device electronics and/or power source, and the like. The device electronics may include device modules (e.g., hardware/software), such as: a processing module 730-a, a memory 715, a communication module 720-a, a power module 725, and the like. The device electronics may also include one or more sensors. Example sensors may include one or more temperature sensors 740, a PPG sensor assembly (e.g., PPG system 735), and one or more motion sensors 745.

The sensors may include associated modules configured to communicate with the respective components/modules of the ring 704, and generate signals associated with the respective sensors. In some aspects, some or all of the components/modules of the ring 704 may be communicatively coupled to one another via wired or wireless connections. Moreover, the ring 704 may include additional and/or alternative sensors or other components that are configured to collect physiological data from the user, including light sensors (e.g., LEDs), oximeters, and the like.

The ring 704 shown and described with reference to FIG. 7B is provided solely for illustrative purposes. As such, the ring 704 may include additional and/or different components as those illustrated in FIG. 7B. Other rings 704 that provide functionality described herein may be fabricated. For example, rings 704 with fewer components (e.g., sensors) may be fabricated. In a specific example, a ring 704 with a single temperature sensor 740 (or other sensor), a power source, and device electronics configured to read the single temperature sensor 740 (or other sensor) may be fabricated. In another specific example, a temperature sensor 740 (or other sensor) may be coupled to a user's finger (e.g., using adhesives, wraps, clamps, spring loaded clamps, etc.). In this case, the sensor may be wired to another computing device, such as a wrist worn computing device that reads the temperature sensor 740 (or other sensor). In other examples, a ring 704 that includes additional sensors and processing functionality may be fabricated.

The housing 705 may include the outer housing 705-b component (e.g., a shell) and the inner housing 705-a component (e.g., a molding). The housing 705 may include additional components (e.g., additional layers). For example, in some implementations, the ring 704 may include one or more insulating layers that electrically insulate the device electronics and other conductive materials (e.g., electrical traces) from the outer housing 705-b (e.g., a metal outer housing 705-b). The housing 705 may provide structural support for the device electronics, battery 712, substrate(s), and other components. For example, the housing 705 may protect the device electronics, battery 712, and substrate(s) from mechanical forces, such as pressure and impacts. The housing 705 may also protect the device electronics, battery 712, and substrate(s) from water and/or other chemicals, as well as provide a barrier against liquid ingress.

The outer housing 705-b may be fabricated from one or more materials. In some implementations, the outer housing 705-b may include a metal, such as titanium, that may provide strength and abrasion resistance at a relatively light weight. The outer housing 705-b may also be fabricated from other materials, such polymers. In some implementations, the outer housing 705-b may be protective as well as decorative.

The inner housing 705-a may be configured to interface with the user's finger. The inner housing 705-a may be formed from a polymer (e.g., a medical grade polymer) or other material. In some implementations, the inner housing 705-a may be transparent. For example, the inner housing 705-a may be transparent or translucent with respect to light emitted by the PPG light emitting diodes (LEDs). In some implementations, the inner housing 705-a component may be molded onto the outer housing 705-b. For example, the inner housing 705-a may include a polymer that is molded (e.g., injection molded) to fit into an outer housing 705-b metallic shell.

The ring 704 may include one or more substrates (not illustrated). The device electronics and battery 712 may be included on the one or more substrates. For example, the device electronics and battery 712 may be mounted on one or more substrates. Example substrates may include one or more PCBs, such as flexible PCB (e.g., polyimide). In some implementations, the electronics/battery 712 may include surface mounted devices (e.g., surface- mount technology (SMT) devices) on a flexible PCB. In some implementations, the one or more substrates (e.g., one or more flexible PCBs) may include electrical traces that provide electrical communication between device electronics. The electrical traces may also connect the battery 712 to the device electronics.

The device electronics, battery 712, and substrates may be arranged in the ring 704 in a variety of ways. In some implementations, one substrate that includes device electronics may be mounted along the bottom of the ring 704 (e.g., the bottom half), such that the sensors (e.g., PPG system 735, temperature sensors 740, motion sensors 745, and other sensors) interface with the underside of the user's finger. In these implementations, the battery 712 may be included along the top portion of the ring 704 (e.g., on another substrate).

The various components/modules of the ring 704 represent functionality (e.g., circuits and other components) that may be included in the ring 704. Modules may include any discrete and/or integrated electronic circuit components that implement analog and/or digital circuits capable of producing the functions attributed to the modules herein. For example, the modules may include analog circuits (e.g., amplification circuits, filtering circuits, analog/digital conversion circuits, and/or other signal conditioning circuits). The modules may also include digital circuits (e.g., combinational or sequential logic circuits, memory circuits etc.).

The memory 715 (memory module) of the ring 704 may include any volatile, non- volatile, magnetic, or electrical media, such as a random access memory (RAM), read-only memory (ROM), non-volatile RAM (NVRAM), electrically-erasable programmable ROM (EEPROM), flash memory, or any other memory device. The memory 715 may store any of the data described herein. For example, the memory 715 may be configured to store data (e.g., motion data, temperature data, PPG data) collected by the respective sensors and PPG system 735. Furthermore, memory 715 may include instructions that, when executed by one or more processing circuits, cause the modules to perform various functions attributed to the modules herein. The device electronics of the ring 704 described herein are only example device electronics. As such, the types of electronic components used to implement the device electronics may vary based on design considerations.

The functions attributed to the modules of the ring 704 described herein may be embodied as one or more processors, hardware, firmware, software, or any combination thereof. Depiction of different features as modules is intended to highlight different functional aspects and does not necessarily imply that such modules must be realized by separate hardware/software components. Rather, functionality associated with one or more modules may be performed by separate hardware/software components or integrated within common hardware/software components.

The processing module 730-a of the ring 704 may include one or more processors (e.g., processing units), microcontrollers, digital signal processors, systems on a chip (SOCs), and/or other processing devices. The processing module 730-a communicates with the modules included in the ring 704. For example, the processing module 730-a may transmit/receive data to/from the modules and other components of the ring 704, such as the sensors. As described herein, the modules may be implemented by various circuit components. Accordingly, the modules may also be referred to as circuits (e.g., a communication circuit and power circuit).

The processing module 730-a may communicate with the memory 715. The memory 715 may include computer-readable instructions that, when executed by the processing module 730-a, cause the processing module 730-a to perform the various functions attributed to the processing module 730-a herein. In some implementations, the processing module 730-a (e.g., a microcontroller) may include additional features associated with other modules, such as communication functionality provided by the communication module 720-a (e.g., an integrated Bluetooth Low Energy transceiver) and/or additional onboard memory 715.

The communication module 720-a may include circuits that provide wireless and/or wired communication with the user device 706 (e.g., communication module 720-b of the user device 706). In some implementations, the communication modules 720-a, 720-b may include wireless communication circuits, such as Bluetooth circuits and/or Wi-Fi circuits. In some implementations, the communication modules 720-a, 720-b can include wired communication circuits, such as Universal Serial Bus (USB) communication circuits. Using the communication module 720-a, the ring 704 and the user device 706 may be configured to communicate with each other. The processing module 730-a of the ring may be configured to transmit/receive data to/from the user device 706 via the communication module 720-a. Example data may include, but is not limited to, motion data, temperature data, pulse waveforms, heart rate data, HRV data, PPG data, and status updates (e.g., charging status, battery charge level, and/or ring 704 configuration settings). The processing module 730-a of the ring may also be configured to receive data from the user device 706, such as updates (e.g., software/firmware updates).

The ring 704 may include a battery 712 (e.g., a rechargeable battery 712). An example battery 712 may include a Lithium-Ion or Lithium-Polymer type battery 712, although a variety of battery 712 options are possible. The battery 712 may be wirelessly charged. In some implementations, the ring 704 may include a power source other than the battery 712, such as a capacitor. The power source (e.g., battery 712 or capacitor) may have a curved geometry that matches the curve of the ring 704. In some aspects, a charger or other power source may include additional sensors that may be used to collect data in addition to, or that supplements, data collected by the ring 704 itself. Moreover, a charger or other power source for the ring 704 may function as a user device 706, in which case the charger or other power source for the ring 704 may be configured to receive data from the ring 704, store and/or process data received from the ring 704, and communicate data between the ring 704 and the servers 710.

In some aspects, the ring 704 includes a power module 725 that may control charging of the battery 712. For example, the power module 725 may interface with an external wireless charger that charges the battery 712 when interfaced with the ring 704. The charger may include a datum structure that mates with a ring 704 datum structure to create a specified orientation with the ring 704 during charging. The power module 725 may also regulate voltage(s) of the device electronics, regulate power output to the device electronics, and monitor the state of charge of the battery 712. In some implementations, the battery 712 may include a protection circuit module (PCM) that protects the battery 712 from high current discharge, over voltage during charging, and under voltage during discharge.

The one or more temperature sensors 740 may be electrically coupled to the processing module 730-a. The temperature sensor 740 may be configured to generate a temperature signal (e.g., temperature data) that indicates a temperature read or sensed by the temperature sensor 740. The processing module 730-a may determine a temperature of the user in the location of the temperature sensor 740. For example, in the ring 704, temperature data generated by the temperature sensor 740 may indicate a temperature of a user at the user's finger (e.g., skin temperature). In some implementations, the temperature sensor 740 may contact the user's skin. In other implementations, a portion of the housing 705 (e.g., the inner housing 705-a) may form a barrier (e.g., a thin, thermally conductive barrier) between the temperature sensor 740 and the user's skin. In some implementations, portions of the ring 704 configured to contact the user's finger may have thermally conductive portions and thermally insulative portions. The thermally conductive portions may conduct heat from the user's finger to the temperature sensors 740. The thermally insulative portions may insulate portions of the ring 704 (e.g., the temperature sensor 740) from ambient temperature.

In some implementations, the temperature sensor 740 may generate a digital signal (e.g., temperature data) that the processing module 730-a may use to determine the temperature. As another example, in cases where the temperature sensor 740 includes a passive sensor, the processing module 730-a (or a temperature sensor 740 module) may measure a current and/or voltage generated by the temperature sensor 740 and determine the temperature based on the measured current and/or voltage. Example temperature sensors 740 may include a thermistor, such as a negative temperature coefficient (NTC) thermistor, or other types of sensors including resistors, transistors, diodes, and/or other electrical/electronic components.

The processing module 730-a may sample the user's temperature over time. For example, the processing module 730-a may sample the user's temperature according to a sampling rate. An example sampling rate may include one sample per second, although the processing module 730-a may be configured to sample the temperature signal at other sampling rates that are higher or lower than one sample per second. In some implementations, the processing module 730-a may sample the user's temperature continuously throughout the day and night. Sampling at a sufficient rate (e.g., one sample per second) throughout the day may provide sufficient temperature data for analysis described herein.

The processing module 730-a may store the sampled temperature data in memory 715. In some implementations, the processing module 730-a may process the sampled temperature data. For example, the processing module 730-a may determine average temperature values over a period of time. In one example, the processing module 730-a may determine an average temperature value each minute by summing all temperature values collected over the minute and dividing by the number of samples over the minute. In a specific example where the temperature is sampled at one sample per second, the average temperature may be a sum of all sampled temperatures for one minute divided by sixty seconds. The memory 715 may store the average temperature values over time. In some implementations, the memory 715 may store average temperatures (e.g., one per minute) instead of sampled temperatures in order to conserve memory 715.

The sampling rate, which may be stored in memory 715, may be configurable. In some implementations, the sampling rate may be the same throughout the day and night. In other implementations, the sampling rate may be changed throughout the day/night. In some implementations, the ring 704 may filter/reject temperature readings, such as large spikes in temperature that are not indicative of physiological changes (e.g., a temperature spike from a hot shower). In some implementations, the ring 704 may filter/reject temperature readings that may not be reliable due to other factors, such as excessive motion during exercise (e.g., as indicated by a motion sensor 745).

The ring 704 (e.g., communication module) may transmit the sampled and/or average temperature data to the user device 706 for storage and/or further processing. The user device 706 may transfer the sampled and/or average temperature data to the server 710 for storage and/or further processing.

Although the ring 704 is illustrated as including a single temperature sensor 740, the ring 704 may include multiple temperature sensors 740 in one or more locations, such as arranged along the inner housing 705-a near the user's finger. In some implementations, the temperature sensors 740 may be stand-alone temperature sensors 740. Additionally, or alternatively, one or more temperature sensors 740 may be included with other components (e.g., packaged with other components), such as with the accelerometer and/or processor.

The processing module 730-a may acquire and process data from multiple temperature sensors 740 in a similar manner described with respect to a single temperature sensor 740. For example, the processing module 730 may individually sample, average, and store temperature data from each of the multiple temperature sensors 740. In other examples, the processing module 730-a may sample the sensors at different rates and average/store different values for the different sensors. In some implementations, the processing module 730-a may be configured to determine a single temperature based on the average of two or more temperatures determined by two or more temperature sensors 740 in different locations on the finger.

The temperature sensors 740 on the ring 704 may acquire distal temperatures at the user's finger (e.g., any finger). For example, one or more temperature sensors 740 on the ring 704 may acquire a user's temperature from the underside of a finger or at a different location on the finger. In some implementations, the ring 704 may continuously acquire distal temperature (e.g., at a sampling rate). Although distal temperature measured by a ring 704 at the finger is described herein, other devices may measure temperature at the same/different locations. In some cases, the distal temperature measured at a user's finger may differ from the temperature measured at a user's wrist or other external body location. Additionally, the distal temperature measured at a user's finger (e.g., a "shell" temperature) may differ from the user's core temperature. As such, the ring 704 may provide a useful temperature signal that may not be acquired at other internal/external locations of the body. In some cases, continuous temperature measurement at the finger may capture temperature fluctuations (e.g., small or large fluctuations) that may not be evident in core temperature. For example, continuous temperature measurement at the finger may capture minute-to-minute or hour-to-hour temperature fluctuations that provide additional insight that may not be provided by other temperature measurements elsewhere in the body.

The ring 704 may include a PPG system 735. The PPG system 735 may include one or more optical transmitters that transmit light. The PPG system 735 may also include one or more optical receivers that receive light transmitted by the one or more optical transmitters. An optical receiver may generate a signal (hereinafter "PPG" signal) that indicates an amount of light received by the optical receiver. The optical transmitters may illuminate a region of the user's finger. The PPG signal generated by the PPG system 735 may indicate the perfusion of blood in the illuminated region. For example, the PPG signal may indicate blood volume changes in the illuminated region caused by a user's pulse pressure. The processing module 730-a may sample the PPG signal and determine a user's pulse waveform based on the PPG signal. The processing module 730-a may determine a variety of physiological parameters based on the user's pulse waveform, such as a user's respiratory rate, heart rate, HRV, oxygen saturation, and other circulatory parameters.

In some implementations, the PPG system 735 may be configured as a reflective PPG system 735 where the optical receiver(s) receive transmitted light that is reflected through the region of the user's finger. In some implementations, the PPG system 735 may be configured as a transmissive PPG system 735 where the optical transmitter(s) and optical receiver(s) are arranged opposite to one another, such that light is transmitted directly through a portion of the user's finger to the optical receiver(s).

The number and ratio of transmitters and receivers included in the PPG system 735 may vary. Example optical transmitters may include light-emitting diodes (LEDs). The optical transmitters may transmit light in the infrared spectrum and/or other spectrums. Example optical receivers may include, but are not limited to, photosensors, phototransistors, and photodiodes. The optical receivers may be configured to generate PPG signals in response to the wavelengths received from the optical transmitters. The location of the transmitters and receivers may vary. Additionally, a single device may include reflective and/or transmissive PPG systems 735.

The PPG system 735 illustrated in FIG. 7B may include a reflective PPG system 735 in some implementations. In these implementations, the PPG system 735 may include a centrally located optical receiver (e.g., at the bottom of the ring 704) and two optical transmitters located on each side of the optical receiver. In this implementation, the PPG system 735 (e.g., optical receiver) may generate the PPG signal based on light received from one or both of the optical transmitters. In other implementations, other placements, combinations, and/or configurations of one or more optical transmitters and/or optical receivers are contemplated.

The processing module 730-a may control one or both of the optical transmitters to transmit light while sampling the PPG signal generated by the optical receiver. In some implementations, the processing module 730-a may cause the optical transmitter with the stronger received signal to transmit light while sampling the PPG signal generated by the optical receiver. For example, the selected optical transmitter may continuously emit light while the PPG signal is sampled at a sampling rate (e.g., 250 Hz).

Sampling the PPG signal generated by the PPG system 735 may result in a pulse waveform that may be referred to as a "PPG." The pulse waveform may indicate blood pressure over time for multiple cardiac cycles. The pulse waveform may include peaks that indicate cardiac cycles. Additionally, the pulse waveform may include respiratory induced variations that may be used to determine respiration rate. The processing module 730-a may store the pulse waveform in memory 715 in some implementations. The processing module 730-a may process the pulse waveform as it is generated and/or from memory 715 to determine user physiological parameters described herein.

The processing module 730-a may determine the user's heart rate based on the pulse waveform. For example, the processing module 730-a may determine heart rate (e.g., in beats per minute) based on the time between peaks in the pulse waveform. The time between peaks may be referred to as an interbeat interval (IBI). The processing module 730-amay store the determined heart rate values and IBI values in memory 715.

The processing module 730-a may determine HRV over time. For example, the processing module 730-a may determine HRV based on the variation in the IBIs. The processing module 730-a may store the HRV values over time in the memory 715. Moreover, the processing module 730-a may determine the user's respiratory rate over time. For example, the processing module730-a may determine respiratory rate based on frequency modulation, amplitude modulation, or baseline modulation of the user's IBI values over a period of time. Respiratory rate may be calculated in breaths per minute or as another breathing rate (e.g., breaths per 30 seconds). The processing module 730-a may store user respiratory rate values over time in the memory 715.

The ring 704 may include one or more motion sensors 745, such as one or more accelerometers (e.g., 6-D accelerometers) and/or one or more gyroscopes (gyros). The motion sensors 745 may generate motion signals that indicate motion of the sensors. For example, the ring 704 may include one or more accelerometers that generate acceleration signals that indicate acceleration of the accelerometers. As another example, the ring 704 may include one or more gyro sensors that generate gyro signals that indicate angular motion (e.g., angular velocity) and/or changes in orientation. The motion sensors 745 may be included in one or more sensor packages. An example accelerometer/gyro sensor is a BOSCH® BM1160 inertial micro electro- mechanical system (MEMS) sensor that may measure angular rates and accelerations in three perpendicular axes.

The processing module 730-a may sample the motion signals at a sampling rate (e.g., 50Hz) and determine the motion of the ring 704 based on the sampled motion signals. For example, the processing module730-a may sample acceleration signals to determine acceleration of the ring 704. As another example, the processing module 730-a may sample a gyro signal to determine angular motion. In some implementations, the processing module 730-a may store motion data in memory 715. Motion data may include sampled motion data as well as motion data that is calculated based on the sampled motion signals (e.g., acceleration and angular values).

The ring 704 may store a variety of data described herein. For example, the ring 704 may store temperature data, such as raw sampled temperature data and calculated temperature data (e.g., average temperatures). As another example, the ring 704 may store PPG signal data, such as pulse waveforms and data calculated based on the pulse waveforms (e.g., heart rate values, IBI values, HRV values, and respiratory rate values). The ring 704 may also store motion data, such as sampled motion data that indicates linear and angular motion.

The ring 704, or other computing device, may calculate and store additional values based on the sampled/calculated physiological data. For example, the processing module 730 may calculate and store various metrics, such as sleep metrics (e.g., a Sleep Score), activity metrics, and readiness metrics. In some implementations, additional values/metrics may be referred to as "derived values." The ring 704, or other computing/wearable device, may calculate a variety of values/metrics with respect to motion. Example derived values for motion data may include, but are not limited to, motion count values, regularity values, intensity values, metabolic equivalence of task values (METs), and orientation values. Motion counts, regularity values, intensity values, and METs may indicate an amount of user motion (e.g., velocity/acceleration) over time. Orientation values may indicate how the ring 704 is oriented on the user's finger and if the ring 704 is worn on the left hand or right hand.

In some implementations, motion counts and regularity values may be determined by counting a number of acceleration peaks within one or more periods of time (e.g., one or more 30 second to 1 minute periods). Intensity values may indicate a number of movements and the associated intensity (e.g., acceleration values) of the movements. The intensity values may be categorized as low, medium, and high, depending on associated threshold acceleration values. METs may be determined based on the intensity of movements during a period of time (e.g., 30 seconds), the regularity/irregularity of the movements, and the number of movements associated with the different intensities.

In some implementations, the processing module 730-a may compress the data stored in memory 715. For example, the processing module 730-a may delete sampled data after making calculations based on the sampled data. As another example, the processing module 730-a may average data over longer periods of time in order to reduce the number of stored values. In a specific example, if average temperatures for a user over one minute are stored in memory 715, the processing module 730-a may calculate average temperatures over a five minute time period for storage, and then subsequently erase the one minute average temperature data. The processing module 730-a may compress data based on a variety of factors, such as the total amount of used/available memory 715 and/or an elapsed time since the ring 704 last transmitted the data to the user device 706.

Although a user's physiological parameters may be measured by sensors included on a ring 704, other devices may measure a user's physiological parameters. For example, although a user's temperature may be measured by a temperature sensor 740 included in a ring 704, other devices may measure a user's temperature. In some examples, other wearable devices (e.g., wrist devices) may include sensors that measure user physiological parameters. Additionally, medical devices, such as external medical devices (e.g., wearable medical devices) and/or implantable medical devices, may measure a user's physiological parameters. One or more sensors on any type of computing device may be used to implement the techniques described herein.

The physiological measurements may be taken continuously throughout the day and/or night. In some implementations, the physiological measurements may be taken during portions of the day and/or portions of the night. In some implementations, the physiological measurements may be taken in response to determining that the user is in a specific state, such as an active state, resting state, and/or a sleeping state. For example, the ring 704 can make physiological measurements in a resting/sleep state in order to acquire cleaner physiological signals. In one example, the ring 704 or other device/system may detect when a user is resting and/or sleeping and acquire physiological parameters (e.g., temperature) for that detected state. The devices/systems may use the resting/sleep physiological data and/or other data when the user is in other states in order to implement the techniques of the present disclosure.

In some implementations, as described previously herein, the ring 704 may be configured to collect, store, and/or process data, and may transfer any of the data described herein to the user device 706 for storage and/or processing. In some aspects, the user device 706 includes a wearable application 750, an operating system (OS), a web browser application (e.g., web browser 780), one or more additional applications, and a GUI 775. The user device 706 may further include other modules and components, including sensors, audio devices, haptic feedback devices, and the like. The wearable application 750 may include an example of an application (e.g., "app") that may be installed on the user device 706. The wearable application 750 may be configured to acquire data from the ring 704, store the acquired data, and process the acquired data as described herein. For example, the wearable application 750 may include a user interface (UI) module 755, an acquisition module 760, a processing module 730-b, a communication module 720-b, and a storage module (e.g., database 765) configured to store application data.

In some cases, the wearable device 704 and the user device 706 may be included within (or make up) the same device. For example, in some cases, the wearable device 704 may be configured to execute the wearable application 750, and may be configured to display data via the GUI 775.

The various data processing operations described herein may be performed by the ring 704, the user device 706, the servers 710, or any combination thereof. For example, in some cases, data collected by the ring 704 may be pre-processed and transmitted to the user device 706. In this example, the user device 706 may perform some data processing operations on the received data, may transmit the data to the servers 710 for data processing, or both. For instance, in some cases, the user device 706 may perform processing operations that require relatively low processing power and/or operations that require a relatively low latency, whereas the user device 706 may transmit the data to the servers 710 for processing operations that require relatively high processing power and/or operations that may allow relatively higher latency.

In some aspects, the ring 704, user device 706, and server 710 of the system 790 may be configured to evaluate sleep patterns for a user. In particular, the respective components of the system 790 may be used to collect data from a user via the ring 704, and generate one or more scores (e.g., Sleep Score, Readiness Score) for the user based on the collected data. For example, as noted previously herein, the ring 704 of the system 790 may be worn by a user to collect data from the user, including temperature, heart rate, HRV, and the like. Data collected by the ring 704 may be used to determine when the user is asleep in order to evaluate the user's sleep for a given "sleep day." In some aspects, scores may be calculated for the user for each respective sleep day, such that a first sleep day is associated with a first set of scores, and a second sleep day is associated with a second set of scores. Scores may be calculated for each respective sleep day based on data collected by the ring 704 during the respective sleep day. Scores may include, but are not limited to, Sleep Scores, Readiness Scores, and the like.

In some cases, "sleep days" may align with the traditional calendar days, such that a given sleep day runs from midnight to midnight of the respective calendar day. In other cases, sleep days may be offset relative to calendar days. For example, sleep days may run from 6:00 pm (18:00) of a calendar day until 6:00 pm (18:00) of the subsequent calendar day. In this example, 6:00 pm may serve as a "cut-off time," where data collected from the user before 6:00 pm is counted for the current sleep day, and data collected from the user after 6:00 pm is counted for the subsequent sleep day. Due to the fact that most individuals sleep the most at night, offsetting sleep days relative to calendar days may enable the system 790 to evaluate sleep patterns for users in such a manner that is consistent with their sleep schedules. In some cases, users may be able to selectively adjust (e.g., via the GUI) a timing of sleep days relative to calendar days so that the sleep days are aligned with the duration of time that the respective users typically sleep.

In some implementations, each overall score for a user for each respective day (e.g., Sleep Score, Readiness Score) may be determined/calculated based on one or more "contributors," "factors," or "contributing factors." For example, a user's overall Sleep Score may be calculated based on a set of contributors, including: total sleep, efficiency, restfulness, REM sleep, deep sleep, latency, timing, or any combination thereof. The Sleep Score may include any quantity of contributors. The "total sleep" contributor may refer to the sum of all sleep periods of the sleep day. The "efficiency" contributor may reflect the percentage of time spent asleep compared to time spent awake while in bed, and may be calculated using the efficiency average of long sleep periods (e.g., primary sleep period) of the sleep day, weighted by a duration of each sleep period. The "restfulness" contributor may indicate how restful the user's sleep is, and may be calculated using the average of all sleep periods of the sleep day, weighted by a duration of each period. The restfulness contributor may be based on a "wake up count" (e.g., sum of all the wake-ups (when user wakes up) detected during different sleep periods), excessive movement, and a "got up count" (e.g., sum of all the got-ups (when user gets out of bed) detected during the different sleep periods).

The "REM sleep" contributor may refer to a sum total of REM sleep durations across all sleep periods of the sleep day including REM sleep. Similarly, the "deep sleep" contributor may refer to a sum total of deep sleep durations across all sleep periods of the sleep day including deep sleep. The "latency" contributor may signify how long (e.g., average, median, longest) the user takes to go to sleep, and may be calculated using the average of long sleep periods throughout the sleep day, weighted by a duration of each period and the number of such periods (e.g., consolidation of a given sleep stage or sleep stages may be its own contributor or weight other contributors). Lastly, the "timing" contributor may refer to a relative timing of sleep periods within the sleep day and/or calendar day, and may be calculated using the average of all sleep periods of the sleep day, weighted by a duration of each period.

By way of another example, a user's overall Readiness Score may be calculated based on a set of contributors, including: sleep, sleep balance, heart rate, HRV balance, recovery index,temperature, activity, activity balance, or any combination thereof. The Readiness Score may include any quantity of contributors. The "sleep" contributor may refer to the combined Sleep Score of all sleep periods within the sleep day. The "sleep balance" contributor may refer to a cumulative duration of all sleep periods within the sleep day. In particular, sleep balance may indicate to a user whether the sleep that the user has been getting over some duration of time (e.g., the past two weeks) is in balance with the user's needs. Typically, adults need 7-9 hours of sleep a night to stay healthy, alert, and to perform at their best both mentally and physically. However, it is normal to have an occasional night of bad sleep, so the sleep balance contributor takes into account long-term sleep patterns to determine whether each user's sleep needs are being met. The "resting heart rate" contributor may indicate a lowest heart rate from the longest sleep period of the sleep day (e.g., primary sleep period) and/or the lowest heart rate from naps occurring after the primary sleep period.

Continuing with reference to the "contributors" (e.g., factors, contributing factors) of the Readiness Score, the "HRV balance" contributor may indicate a highest HRV average from the primary sleep period and the naps happening after the primary sleep period. The HRV balance contributor may help users keep track of their recovery status by comparing their HRV trend over a first time period (e.g., two weeks) to an average HRV over some second, longer time period (e.g., three months). The "recovery index" contributor may be calculated based on the longest sleep period. Recovery index measures how long it takes for a user's resting heart rate to stabilize during the night. A sign of a very good recovery is that the user's resting heart rate stabilizes during the first half of the night, at least six hours before the user wakes up, leaving the body time to recover for the next day. The "body temperature" contributor may be calculated based on the longest sleep period (e.g., primary sleep period) or based on a nap happening after the longest sleep period if the user's highest temperature during the nap is at least 0.5°C higher (or another value) than the highest temperature during the longest period. In some aspects, the ring may measure a user's body temperature while the user is asleep, and the system 790 may display the user's average temperature relative to the user's baseline temperature. If a user's body temperature is outside of their normal range (e.g., clearly above or below 0.0 or another value), the body temperature contributor may be highlighted (e.g., go to a "Pay attention" state) or otherwise generate an alert for the user.

What is claimed is:

1. A finger-worn wearable ring device comprising:
a housing having an interior diameter between 12 mm and 24 mm and an exterior diameter between 18 mm and 30 mm for at least a portion of the finger-worn wearable ring device, the housing comprising:
a curved battery;
a printed circuit board comprising a first rigid portion, a second rigid portion, a third rigid portion, a first flexible portion separating the second rigid portion from the first rigid portion, and a second flexible portion separating the third rigid portion from the second rigid portion;
a first light emitting diode (LED) disposed on the first rigid portion of the printed circuit board, the first LED configured to output green light;
a second LED disposed on the first rigid portion of the printed circuit board, the second LED configured to output yellow light;
a third LED disposed on the third rigid portion of the printed circuit board, the third LED configured to output red light; and
a first photodiode disposed on the first rigid portion of the printed circuit board, the first photodiode configured to detect light output from at least one of the first LED, the second LED, or the third LED;
wherein the first LED, the second LED, and the third LED are directly attached to the printed circuit board.

2. The finger-worn wearable ring device of claim 1, further comprising:
a vertical cavity surface emitting laser assembly;
wherein the vertical cavity surface emitting laser assembly is disposed on the second rigid portion of the printed circuit board.

3. The finger-worn wearable ring device of claim 1, further comprising:
a fourth LED disposed on the printed circuit board adjacent to the third LED, the fourth LED configured to output red light;
a first reflective structure disposed on the printed circuit board between the third LED and the fourth LED; and
a second reflective structure disposed on the printed circuit board between the first LED and the second LED;
wherein both the first reflective structure and the second reflective structure comprise a substantially triangular cross-sectional geometry, and wherein both the first reflective structure and the second reflective structure are formed at least partially of metal.

4. The finger-worn wearable ring device of claim 1, wherein the housing comprises an upper half and a lower half;
wherein the first LED is disposed in the upper half of the housing, and the third LED is disposed in the lower half of the housing; and
wherein the battery is disposed in the upper half of the housing.

5. A wearable device comprising:
a printed circuit board comprising a first portion and a second portion;
a first LED disposed on the first portion of the printed circuit board, the first LED configured to output green light;
a second LED disposed on the first portion of the printed circuit board;
a third LED disposed on the second portion of the printed circuit board, the third LED configured to output red light; and
a photodiode disposed on the first portion of the printed circuit board, the photodiode configured to detect light output from at least one of the first LED, the second LED, or the third LED;
wherein the first LED, the second LED, and the third LED are directly attached to the printed circuit board.

6. The wearable device of claim 5, wherein the first portion of the printed circuit board is a first rigid portion and the second portion is a second rigid portion, the printed circuit board further comprising:
a third rigid portion disposed between the first rigid portion and the second rigid portion;
a first flexible portion separating the third rigid portion from the first rigid portion; and
a second flexible portion separating the third rigid portion from the second rigid portion.

7. The wearable device of claim 6, further comprising:
at least one of: a laser diode assembly, or a vertical cavity surface emitting laser assembly.

8. The wearable device of claim 7, wherein the laser diode assembly is disposed on the third rigid portion of the printed circuit board.

9. The wearable device of claim 5, wherein the second LED is configured to output yellow light, the wearable device further comprising:
a fourth LED disposed on the printed circuit board adjacent to the third LED, the fourth LED configured to output red light; and
a first reflective structure disposed on the printed circuit board between the third LED and the fourth LED.

10. The wearable device of claim 9, further comprising:
a second reflective structure disposed on the printed circuit board between the first LED and the second LED.

11. The wearable device of claim 10, wherein both the first reflective structure and the second reflective structure comprise a substantially triangular cross-sectional geometry, and wherein both the first reflective structure and the second reflective structure are formed at least partially of metal.

12. The wearable device of claim 10, wherein the second reflective structure is disposed on a first side of the second LED, the device further comprising:
a third reflective structure disposed on the printed circuit board on a second side of the second LED, wherein the third reflective structure has a different configuration than the second reflective structure.

13. The wearable device of claim 5, further comprising:
a housing comprising an optically clear portion, the optically clear portion having a raised protrusion forming a dome;
wherein light output from the third LED propagates through the dome.

14. The wearable device of claim 5, wherein the first LED, the second LED, and the third LED are chip-level LEDs.

15. The wearable device of claim 5, further comprising:
a housing comprising an upper half and a lower half;
wherein the first LED is disposed in the upper half of the housing, and the third LED is disposed in the lower half of the housing.

16. The wearable device of claim 15, further comprising:
a curved battery disposed in the upper half of the housing.

17. The wearable device of claim 5, wherein the device is a finger-worn wearable device, the finger-worn wearable device further comprising:
a housing having an interior diameter between 12 mm and 24 mm and an exterior diameter between 18 mm and 30 mm for at least a portion of the finger-worn wearable ring device.

18. A wearable device comprising:
a printed circuit board comprising a first portion, a second portion, and a third portion, wherein the second portion is disposed between the first portion and the third portion;
a first light emitting chip disposed on the first portion of the printed circuit board;
a second light emitting chip disposed on the first portion of the printed circuit board;
at least one of: a laser diode assembly configured to output infrared light, or a vertical cavity surface emitting laser assembly, the at least one of the laser diode assembly or the vertical cavity surface emitting laser assembly disposed on the second portion of the printed circuit board; and
a first photodiode disposed on the first portion of the printed circuit board, the photodiode configured to detect light output from at least one of the first light emitting chip or the second light emitting chip;
wherein the first light emitting chip and the second light emitting chip are directly attached to the printed circuit board.

19. The wearable device of claim 18, wherein the printed circuit board further comprises:
a first flexible portion separating the second portion from the first portion; and
a second flexible portion separating the third portion from the second portion.

20. The wearable device of claim 18, further comprising:
a third light emitting chip disposed on the third portion of the printed circuit board;
a fourth light emitting chip disposed adjacent to the third light emitting chip;
a first reflective structure disposed between the third light emitting chip and the fourth light emitting chip; and
a second reflective structure disposed between the first light emitting chip and the second light emitting chip;
wherein both the first reflective structure and the second reflective structure comprise a substantially triangular cross-sectional geometry, and wherein both the first reflective structure and the second reflective structure are formed at least partially of metal.
